SEQUENCE LISTING (30 sequences)

SEQ ID NO: 1 - protein, 343 aa, synthetic construct
  REGION 1..343: note = CIndR7
MLLLVTSLLLCELPHPAFLLIPCHPECQPQNGSVTCFGPEADQCVACAHYKDPPFCVARCPSGVKPDLSYMPIWKFPDEEGACQPCPINCTHSCVDLDDKGCPAEQRASPLTGGGSGGGSPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 2 - protein, 391 aa, synthetic construct
  REGION 1..391: note = CIndR7/21
MLLLVTSLLLCELPHPAFLLIPCHPECQPQNGSVTCFGPEADQCVACAHYKDPPFCVARCPSGVKPDLSYMPIWKFPDEEGACQPCPINCTHSCVDLDDKGCPAEQRASPLTGGGSGGGSPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGGGGSGGGGSSPVECDFTSPGDEGPPRSYLRQWVVIPPPLSSPGPQAS

SEQ ID NO: 3 - protein, 391 aa, synthetic construct
  REGION 1..391: note = CIndR21
MLLLVTSLLLCELPHPAFLLIPCHPECQPQNGSVTCFGPEADQCVACAHYKDPPFCVARCPSGVKPDLSYMPIWKFPDEEGACQPCPINCTHSCVDLDDKGCPAEQRASPLTGGGSGGGSPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAFVTMSSFYQNQGGGGSGGGGSSPVECDFTSPGDEGPPRSYLRQWVVIPPPLSSPGPQAS

SEQ ID NO: 4 - DNA, 1029 bp, synthetic construct
  misc_feature 1..1029: note = CIndR7
  source 1..1029: mol_type = other DNA
atgctgctgctggtcacatctctgctgctgtgcgagctgccccatcctgcctttctgctgatcccttgtcaccccgagtgccagcctcagaatggcagcgtgacatgctttggccctgaggccgatcagtgtgtggcctgcgctcactacaaggaccctccattctgcgtggccagatgtccttctggcgtgaagcccgacctgagctacatgcccatctggaagttccccgatgaggaaggcgcctgccagccttgtcctatcaattgcacacacagctgcgtggacctggacgacaaaggatgtcctgccgagcagagagcctctccacttactggcggaggaagcggcggaggatctcctatcctgctgacctgtcctacaatcagcatcctgagctttttcagcgtggccctgctcgtgatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagagatgaggtggaaggctttctgcaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagctccagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggacagcccatcctgaccagcctgggcagcaatcaagaagaggcctacgtcaccatgagcagcttctaccagaaccag

SEQ ID NO: 5 - DNA, 1173 bp, synthetic construct
  misc_feature 1..1173: note = CIndR7/21
  source 1..1173: mol_type = other DNA
atgctgctgctggtcacatctctgctgctgtgcgagctgccccatcctgcctttctgctgatcccttgtcaccccgagtgccagcctcagaatggcagcgtgacatgctttggccctgaggccgatcagtgtgtggcctgcgctcactacaaggaccctccattctgcgtggccagatgtccttctggcgtgaagcccgacctgagctacatgcccatctggaagttccccgatgaggaaggcgcctgccagccttgtcctatcaattgcacacacagctgcgtggacctggacgacaaaggatgtcctgccgagcagagagcctctccacttactggcggaggaagcggcggaggatctcctatcctgctgacctgtcctacaatcagcatcctgagctttttcagcgtggccctgctcgtgatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagagatgaggtggaaggctttctgcaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagctccagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggacagcccatcctgaccagcctgggcagcaatcaagaagaggcctacgtcaccatgagcagcttctaccagaaccagggcggaggcggatctggtggtggtggatcttctcccgtggaatgcgacttcacaagccctggcgacgagggacctcctagaagctatctgagacagtgggtcgtgatccctccacctctgtctagtcctggacctcaggcttct

SEQ ID NO: 6 - DNA, 1173 bp, synthetic construct
  misc_feature 1..1173: note = CIndR21
  source 1..1173: mol_type = other DNA
atgctgctgctggtcacatctctgctgctgtgcgagctgccccatcctgcctttctgctgatcccttgtcaccccgagtgccagcctcagaatggcagcgtgacatgctttggccctgaggccgatcagtgtgtggcctgcgctcactacaaggaccctccattctgcgtggccagatgtccttctggcgtgaagcccgacctgagctacatgcccatctggaagttccccgatgaggaaggcgcctgccagccttgtcctatcaattgcacacacagctgcgtggacctggacgacaaaggatgtcctgccgagcagagagcctctccacttactggcggaggaagcggcggaggatctcctatcctgctgacctgtcctacaatcagcatcctgagctttttcagcgtggccctgctcgtgatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagagatgaggtggaaggctttctgcaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagctccagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggacagcccatcctgaccagcctgggcagcaatcaagaagaggccttcgtcaccatgagcagcttctaccagaaccagggcggaggcggatctggtggtggtggatcttctcccgtggaatgcgacttcacaagccctggcgacgagggacctcctagaagctatctgagacagtgggtcgtgatccctccacctctgtctagtcctggacctcaggcttct

SEQ ID NO: 7 - protein, 22 aa, synthetic construct
  REGION 1..22: note = GMCSF signal sequence
MLLLVTSLLLCELPHPAFLLIP

SEQ ID NO: 8 - protein, 98 aa, synthetic construct
  REGION 1..98: note = Her2tG extracellular domain
CHPECQPQNGSVTCFGPEADQCVACAHYKDPPFCVARCPSGVKPDLSYMPIWKFPDEEGACQPCPINCTHSCVDLDDKGCPAEQRASPLTGGGSGGGS

SEQ ID NO: 9 - protein, 28 aa, synthetic construct
  REGION 1..28: note = IL7R transmembrane with dimerizing insertion
PILLTCPTISILSFFSVALLVILACVLW

SEQ ID NO: 10 - protein, 195 aa, synthetic construct
  REGION 1..195: note = IL7R intracellular domain
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 11 - protein, 10 aa, synthetic construct
  REGION 1..10: note = Linker
GGGGSGGGGS

SEQ ID NO: 12 - protein, 38 aa, synthetic construct
  REGION 1..38: note = IL21R-peptide
SPVECDFTSPGDEGPPRSYLRQWVVIPPPLSSPGPQAS

SEQ ID NO: 13 - protein, 195 aa, synthetic construct
  REGION 1..195: note = IL7R intracellular domain with STAT5 signalingmotif ablated
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAFVTMSSFYQNQ

SEQ ID NO: 14 - DNA, 66 bp, synthetic construct
  misc_feature 1..66: note = FIG. 18GMCSF signal sequence
  source 1..66: mol_type = other DNA
atgctgctgctggtcacatctctgctgctgtgcgagctgccccatcctgcctttctgctgatccct

SEQ ID NO: 15 - DNA, 294 bp, synthetic construct
  misc_feature 1..294: note = Her2tG extracellular domain
  source 1..294: mol_type = other DNA
tgtcaccccgagtgccagcctcagaatggcagcgtgacatgctttggccctgaggccgatcagtgtgtggcctgcgctcactacaaggaccctccattctgcgtggccagatgtccttctggcgtgaagcccgacctgagctacatgcccatctggaagttccccgatgaggaaggcgcctgccagccttgtcctatcaattgcacacacagctgcgtggacctggacgacaaaggatgtcctgccgagcagagagcctctccacttactggcggaggaagcggcggaggatct

SEQ ID NO: 16 - DNA, 84 bp, synthetic construct
  misc_feature 1..84: note = IL7R transmembrane with dimerizing insertion
  source 1..84: mol_type = other DNA
cctatcctgctgacctgtcctacaatcagcatcctgagctttttcagcgtggccctgctcgtgatcctggcctgtgtgctgtgg

SEQ ID NO: 17 - DNA, 585 bp, synthetic construct
  misc_feature 1..585: note = IL7R intracellular domain
  source 1..585: mol_type = other DNA
aagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagagatgaggtggaaggctttctgcaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagctccagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggacagcccatcctgaccagcctgggcagcaatcaagaagaggcctacgtcaccatgagcagcttctaccagaaccag

SEQ ID NO: 18 - DNA, 30 bp, synthetic construct
  misc_feature 1..30: note = Linker
  source 1..30: mol_type = other DNA
ggcggaggcggatctggtggtggtggatct

SEQ ID NO: 19 - DNA, 114 bp, synthetic construct
  misc_feature 1..114: note = IL21R-peptide
  source 1..114: mol_type = other DNA
tctcccgtggaatgcgacttcacaagccctggcgacgagggacctcctagaagctatctgagacagtgggtcgtgatccctccacctctgtctagtcctggacctcaggcttct

SEQ ID NO: 20 - DNA, 585 bp, synthetic construct
  misc_feature 1..585: note = IL7R intracellular domain with STAT5 signalingmotif ablated
  source 1..585: mol_type = other DNA
aagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagagatgaggtggaaggctttctgcaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagctccagaagcctggattgcagagagagcggcaagaacggccctcacgtgtaccaggatctgctcctgtctctgggcaccaccaatagcacactgcctccaccattcagcctgcagagcggcatcctgacactgaaccctgttgctcagggacagcccatcctgaccagcctgggcagcaatcaagaagaggccttcgtcaccatgagcagcttctaccagaaccag

SEQ ID NO: 21 - DNA, 347 bp, synthetic construct
  misc_feature 1..347: note = MND promoter
  source 1..347: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 22 - DNA, 135 bp, synthetic construct
  misc_feature 1..135: note = RBG PolyA
  source 1..135: mol_type = other DNA
ttttccctctgccaaaaattatggggacatcatgaagccccttgagcatctgacttctggctaataaaggaaatttattttcattgcaatagtgtgttggaattttttgtgtctctcactcggaaggacataagg

SEQ ID NO: 23-24 - skipped (2 entries)

SEQ ID NO: 25 - DNA, 10 bp, synthetic construct
  misc_feature 1..10: note = Linker
  source 1..10: mol_type = other DNA
ggggsggggs

SEQ ID NO: 26 - DNA, 15 bp, synthetic construct
  misc_feature 1..15: note = Linker
  source 1..15: mol_type = other DNA
ggggsggggsggggs

SEQ ID NO: 27 - protein, 462 aa, synthetic construct
  REGION 1..462: note = CD19CAR
MLLLVTSLLLCELPHPAFLLIPEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 323 aa, synthetic construct
  REGION 1..323: note = CD19t
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 29 - DNA, 1386 bp, synthetic construct
  misc_feature 1..1386: note = anti-CD19CAR
  source 1..1386: mol_type = other DNA
atgctgctgctggtcacatctctgctgctgtgcgagctgccccatcctgcctttctgctgattcctgaggtgcagctggtggaatctggcggaggacttgtgcagcctggcagaagcctgagactgtcttgtgccgccagcggcttcaccttcgacgattatgccatgcactgggtccgacaggcccctggcaaaggacttgaatgggtgtccggcatcagctggaacagcggcagaatcggctatgccgacagcgtgaagggcagattcaccatcagccgggacaacgccaagaacagcctgttcctgcagatgaactccctgagagccgaggacaccgccgtgtactactgtgccagagatcagggctaccactactacgactctgccgagcacgccttcgatatctggggccagggaacagtggtcaccgtttctagcggaggcggaggatctggtggtggtggctctggtggcggcggatctcaatctgctctgacccagcctagaagcgtgtccggctttcctggccagagcgtgacaatcagctgtaccggcaccaccagcgacgacgtgtcatggtatcagcagcaccctggaaaggcccctcagctcatgctgtacgatgtgtccaaaagacccagcggcgtgccccacagattttccggaagtagatctggcagagccgccagcctgatcatctctggactgcagacagaggacgaggccgactacttctgcagcagctatgccggcagatacaacagcgtgctgtttggcggaggcaccaagctgaccgtgctggaatctaagtacggccctccttgtcctccatgtcctatgttctgggtgctcgtggttgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgcagctgtcggttccccgaggaagaagaaggcggctgcgagctgagagtgaagttcagcagatccgctgacgcccctgcctatcagcagggacagaaccagctgtataacgagctgaacctggggcgcagagaagagtacgacgtgctggacaagagaagaggcagggaccctgagatgggcggcaagcccagaagaaagaaccctcaagagggcctctacaacgagctgcagaaagacaagatggccgaggcctacagcgagatcggaatgaagggcgaacgcagaagaggaaagggccacgacggactgtatcagggactgagcaccgccaccaaggacacctatgatgccctgcacatgcaggccctgcctccaaga

SEQ ID NO: 30 - DNA, 969 bp, synthetic construct
  misc_feature 1..969: note = CD19t
  source 1..969: mol_type = other DNA
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaaga

(19) United States
(12) Patent Application Publication
(10) Pub. No.: US 2025/0206804 A1
(43) Pub. Date: Jun. 26, 2025

(54) CHIMERIC CYTOKINE RECEPTORS

(71) Applicant: Seattle Children's Hospital (dba Seattle Children's Research Institute), Seattle, WA (US)

(72) Inventors: Christopher P. Saxby, Seattle, WA (US); Michael C. Jensen, Seattle, WA (US)

(21) Appl. No.: 18/852,047

(22) PCT Filed: Mar. 30, 2023
(86) PCT No.: PCT/US2023/065157

§ 371 Date: Sep. 27, 2024

Related U.S. Application Data
(60) Provisional application No. 63/362,368, filed on Apr. 1, 2022.

(51) Int. Cl. C07K 14/715; C07K 14/705; C07K 14/71; C07K 14/82

(52) U.S. Cl. CPC C07K 14/7155; C07K 14/70503; C07K 14/71; C07K 14/82; C07K 2319/02; C07K 2319/03

(57) ABSTRACT

Some embodiments of the methods and compositions provided herein relate to chimeric cytokine receptors. In some embodiments, a chimeric cytokine receptor can include an IL-7 domain and/or an IL-21 domain. In some embodiments, the chimeric receptor can include a hybrid IL- 7 and IL-21 cytokine receptor. In some embodiments, the chimeric receptor is capable of combinatorial interleukin signaling when combined with a T cell. In some embodiments, a T cell containing a chimeric cytokine receptor can be readily activated and/or expanded in the absence of an exogenous cytokine.

### CROSS-REFERENCE TO RELATED APPLICATIONS

This application claims priority to U.S. Prov. App. No. 63/362,368 filed Apr. 1, 2022 entitled “CHIMERIC CYTOKINE RECEPTORS” the disclosure of which is expressly incorporated by reference herein in its entirety.

### REFERENCE TO SEQUENCE LISTING

The present application is being filed along with a Sequence Listing in electronic format. The Sequence Listing is provided as a file entitled SCRI364WOSEQLISTXML, created Mar. 23, 2023, which is approximately 46,728 bytes in size. The information in the electronic format of the Sequence Listing is incorporated herein by reference in its entirety.

### FIELD

Some embodiments of the methods and compositions provided herein relate to chimeric cytokine receptors. In some embodiments, a chimeric cytokine receptor can include an IL-7 domain and/or an IL-21 domain. In some embodiments, the chimeric receptor can include a hybrid IL-7 and IL-21 cytokine receptor. In some embodiments, the chimeric receptor is capable of combinatorial interleukin signaling when combined with a T cell. In some embodiments, a T cell containing a chimeric cytokine receptor can be readily activated and/or expanded in the absence of an exogenous cytokine.

### BACKGROUND

In cell-based adoptive immunotherapy, T cells isolated from a patient can be modified to express synthetic proteins that enable the cells to perform new therapeutic functions after they are subsequently transferred back into the patient. Examples of such synthetic proteins are chimeric antigen receptors (CARs) and engineered T cell Receptors (TCR). An example of a currently used CAR is a fusion of an extracellular recognition domain (e.g., an antigen-binding domain), a transmnembrane domain, and one or more intracellular signaling domains. Upon antigen engagement, the intracellular signaling portion of the CAR can initiate an activation-related response in an immune cell, such as release of cytolytic molecules to induce tumor cell death.

In preclinical models, CAR T cell therapy can be improved by supplementing with gamma chain cytokines, soluble factors that promote T cell growth and survival. However, systemic administration of cytokines to patients is not a therapeutically viable solution, as clinical trials have shown that this approach can lead to toxic side-effects.

### SUMMARY

Disclosed herein is a chimeric cytokine receptor. In some embodiments, the chimeric cytokine receptor comprises (i) an extracellular domain, optionally, wherein the extracellular domain comprises a cell surface selectable polypeptide, optionally, wherein the selectable polypeptide is selected from an extracellular HER2 domain, an extracellular EGFR domain, and an extracellular CD19 domain; (ii) a transmembrane interleukin-7 receptor (IL-7R) domain; and (iii) an intracellular IL-7R domain. In some embodiments, the transmembrane IL-7R domain is modified to promote dimerization of the chimeric receptor, and/or constitutive signaling. In some embodiments, the chimeric cytokine receptor further comprises a GMCSF signal sequence. In some embodiments, the GMCSF signal sequence has at least 80% sequence identity to the amino acid sequence of SEQ ID NO: 7, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 7. In some embodiments, the extracellular domain comprises: (i) the extracellular HER2 domain, optionally wherein the extracellular HER2 domain comprises or consists of a polypeptide having at least 80% sequence identity to the amino acid sequence of SEQ ID NO: 8, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 8; (ii) the extracellular CD19 domain, optionally wherein the extracellular CD19 domain comprises a truncated CD19 polypeptide (CD19t) comprising or consisting of a polypeptide having an amino acid sequence having at least 90% sequence identity to the amino acid sequence of SEQ ID NO:28; or (iii) the extracellular EGFR domain, optionally wherein the extracellular EGFR domain comprises a truncated EGFR polypeptide (EGFRt). In some embodiments, the extracellular HER2 domain lacks domain I, domain II, and domain III of an extracellular HER2 protein. In some embodiments, the transmembrane IL-7R domain has at least 80% sequence identity to the amino acid sequence of SEQ ID NO: 9, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 9. In some embodiments, the transmembrane IL-7R domain is modified to promote dimerization of the chimeric receptor and comprises the amino acid sequence TCP. In some embodiments, the intracellular IL-7R domain has at least 80% sequence identity to the amino acid sequence of SEQ ID NO: 10, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 10. In some embodiments, the chimeric cytokine receptor further comprises an interleukin-21 receptor (IL-21R) polypeptide attached to the C-terminus of the IL-7R domain via a linker. In some embodiments, the linker has at least 80% sequence identity to the amino acid sequence of SEQ ID NO: 11, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 11. In some embodiments, the linker is selected from the group consisting of: SEQ ID NO: 23, SEQ ID NO: 24, SEQ ID NO: 25, and SEQ ID NO: 26. In some embodiments, the linker has a length from 3 amino acids to 30 amino acids. In some embodiments, the IL-21R-peptide has at least 80% sequence identity to the amino acid sequence of SEQ ID NO: 12, such as at least 80%, 81%, 82%, 83%, 84%, 85% 86%, 87%, 88%, 89%, 90%, 91%, 92%, 93%, 94%, 95%, 96%, 97%, 98%, 99%, or 100% sequence identity to the amino acid sequence of SEQ ID NO: 12. In some embodiments, the intracellular IL-7R domain is modified to ablate a STAT5 signaling motif. In some embodiments, the intracellular IL-7R domain has at least 80% identity to the amino acid sequence of SEQ ID NO: 13, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 13. In some embodiments, the intracellular IL-7R domain comprises a Y449F mutation. In some embodiments, the chimeric cytokine receptor comprises an amino acid sequence having at least 80% sequence identity to any one of SEQ ID NO: 1, SEQ ID NO: 2, or SEQ ID NO: 3, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of any one of SEQ ID NO: 1, SEQ ID NO: 2, or SEQ ID NO: 3.

Also disclosed herein is a polynucleotide. In some embodiments, the polynucleotide encodes the chimeric cytokine receptor of any of the embodiments disclosed herein.

Also disclosed herein is a polynucleotide encoding the chimeric cytokine receptor of any one of the present embodiments, wherein the polynucleotide comprises a nucleotide sequence having at least 80% sequence identity to any one of SEQ ID NO: 4, SEQ ID NO: 5, or SEQ ID NO: 6, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO: 4, SEQ ID NO: 5, or SEQ ID NO: 6. In some embodiments, the polynucleotide further comprises a promoter. In some embodiments, the promoter is a constitutive promoter, such as an EF1alpha promoter, or an MIND promoter (e.g., SEQ ID NO: 21). In some embodiments, the polynucleotide further comprises a transcriptional terminator. In some embodiments, the transcriptional terminator is an RBG Poly A sequence (e.g., SEQ ID NO: 22). In some embodiments, the polynucleotide further comprises a translational factor. In some embodiments, the translational factor is EF1a. In some embodiments, the polynucleotide further comprises a nucleic acid encoding a selectable marker, such as a truncated EGFR (EGFRt) polypeptide or a truncated CD19 (CD19t) polypeptide, such as SEQ ID NO: 30. In some embodiments, the polynucleotide further comprises a selection factor and/or resistance cassette. In some embodiments, the selection factor and/or resistance cassette encodes a double mutant dihydrofolate reductase (DHFRdmn) polypeptide. In some embodiments, the polynucleotide further comprises a ribosomal skip sequence and/or a sequence encoding a self cleaving peptide. In some embodiments, the ribosomal skip sequence comprises a T2A skip sequence, and/or the self-cleaving peptide comprises a P2A self-cleaving peptide sequence. In some embodiments, the polynucleotide further comprises a nucleic acid sequence encoding a chimeric antigen receptor (CAR), such an anti-CD19 CAR. In some embodiments, the polynucleotide encodes a chimeric receptor optimized for expression in human cells.

Also disclosed herein is a vector. In some embodiments, the vector comprises the polynucleotide of any of the embodiments disclosed herein. In some embodiments, the vector comprises a viral vector. In some embodiments, the vector is selected from a lentiviral vector, an adeno-associated viral vector, or an adenoviral vector. In some embodiments, the vector comprises a lentiviral vector.

Also disclosed herein is a cell. In some embodiments, the cell comprises one or more of the polypeptides or polynucleotides and/or the vectors disclosed herein. In some embodiments, the cell additionally comprises a polynucleotide encoding a chimeric antigen receptor (CAR). In some embodiments, the cell is a T cell. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian. In some embodiments, the cell is human. In some embodiments, the cell is ex Vivo.

Also disclosed herein are methods of treating, inhibiting, or ameliorating a disease or disorder in a subject. In some embodiments, the method comprises administering the cell of any one of the embodiments disclosed herein to the subject in need thereof. In some embodiments, the disease or disorder is an autoimmune disease. In some embodiments, the disease or disorder is inflammation. In some embodiments, the disease or disorder is a cancer. In some embodiments, the treatment or amelioration of the disease or disorder lacks co-administration of a cytokine to the subject. In some embodiments, the method further comprises co-administering a cytokine to the subject, wherein the dose of the cytokine administered is reduced compared to the dose of the cytokine co-administered to a subject who has been administered a cell comprising a CAR, which lacks the polypeptide of any one of the present embodiments, the polynucleotide of any one of the present embodiments, and/or the vector of any one of the present embodiments disclosed herein. In some embodiments, the cytokine is selected from IL-7 or IL-21. In some embodiments, the cytokine comprises IL-7. In some embodiments, the cytokine comprises IL-21. In some embodiments, the cell is autologous to the subject. In some embodiments, the cancer comprises a solid tumor such as a colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma. In some embodiments, the subject is mammalian. In some embodiments, the subject is human.

Also disclosed herein is a method of preparing a population of cells comprising a chimeric antigen receptor (CAR). In some embodiments, the method comprises (a) transducing a T cell with the polynucleotide of any one of the present embodiments, (b) transducing the T cell with a polynucleotide encoding a CAR, and (c) culturing the transduced T cell under conditions to stimulate activation and expansion of the T cell, wherein the culture media lacks an exogenous cytokine. In some embodiments, step (b) is performed before step (a). In some embodiments, step (a) and (b) are performed concurrently. In some embodiments, the cytokine is selected from IL-7 or IL-21. In some embodiments, the cytokine comprises IL-7. In some embodiments, the cytokine comprises IL-21. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian. In some embodiments, the cell is human. In some embodiments, the cell is ex vivo.

Also disclosed herein is a method of preparing a population of cells comprising a chimeric antigen receptor (CAR). In some embodiments, the method comprises (a) transducing a T cell with the polynucleotide of any one of the present embodiments, (b) transducing the T cell with a polynucleotide encoding a CAR, and (c) culturing the transduced T cell under conditions sufficient to stimulate activation and expansion of the T cell, wherein the culture media comprises a reduced amount of an exogenous cytokine as compared to an amount sufficient to stimulate activation and expansion of the T cell lacking the polynucleotide encoding a chimeric receptor. In some embodiments, step (b) is performed before step (a). In some embodiments, step (a) and (b) are performed concurrently. In some embodiments, the cytokine is selected from IL-7 or IL-21. In some embodiments, the cytokine comprises IL-7. In some embodiments, the cytokine comprises IL-21. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian. In some embodiments, the cell is human. In some embodiments, the cell is ex vivo.

Also disclosed herein is a method of culturing a cell, comprising: (a) obtaining a cell comprising the polynucleotide of any one of the present embodiments; and (b) culturing the cell in a medium lacking an exogenous cytokine or having a reduced amount of the cytokine in the medium, wherein the amount of the cytokine is reduced compared to an amount in a medium in which a cell lacking the polypeptide is cultured; and optionally, wherein the culturing provides a population of cells sufficient for an infusion. In some embodiments, the amount of the cytokine in the medium is sufficient to activate and/or expand the cell. In some embodiments, the cell comprises a chimeric antigen receptor (CAR). In some embodiments, the cytokine is selected from IL-7 or IL-21. In some embodiments, the cytokine comprises IL-7. In some embodiments, the cytokine comprises IL-21. In some embodiments, the cell is a T cell. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian. In some embodiments, the cell is human. In some embodiments, the cell is ex vivo.

### BRIEF DESCRIPTION OF THE DRAWINGS

FIG. 1 (left panel) depicts a schematic, which includes a HER polypeptide with a portion of the extracellular highlighted within a dotted box; a constitutively active IL7R mutant with a transmembrane domain containing a mutation and an intracellular STATS signaling domain each highlighted in dotted boxes; and an IL21 signaling complex with a STAT3 signaling domain highlight in a dotted box. FIG. 1 (right panel) is a schematic that shows an example chimeric receptor (CIndR), which includes the extracellular domain of the HER2; the transmembrane domain containing a mutation and the intracellular STAT5 signaling domain of the IL7R; and the signaling domain of the IL121 signaling complex joined to the rest of the chimeric receptor via a linker.

FIG. 2 (upper panel) depicts a schematic, which includes various chimeric receptors, and a control marker only receptor. FIG. 2 (lower panel) depicts a polynucleotide encoding a CIndR linked to a MND promoter; an EF1-alpha promoter linked to a polynucleotide encoding the anti-CD19 CAR, a T2A sequence, the selectable marker (DHFRdm), a P2A sequence, and the cell surface marker (EGFRt) in a vector.

FIG. 3A depicts a schematic of a chimeric receptor ClndR7. ClndR7 comprises an extracellular domain of a truncated HER2 polypeptide (Her2tG) (1), a transmembrane domain of IL7R with an insertion (2), and an intracellular domain of IL7R (3).

FIG. 3B depicts an alternative schematic of a chimeric receptor ClndR7 with a Y449 residue in the intracellular domain of IL7R (3).

FIG. 3C depicts a schematic of a chimeric receptor ClndR7/21. ClindR7/21 comprises a flexible linker (4), and a IL21R peptide (5).

FIG. 3D depicts a schematic of a chimeric receptor ClndR-7/21 with a Y449 residue in the intracellular domain of IL7R.

FIG. 3E depicts a schematic of a chimeric receptor ClndR21 with a F449 mutation. ClndR21 was generated by ablating the STAT5 signaling motif in the IL7 receptor cytoplasmic domain (6).

FIG. 4 depicts a non-limiting example schematic of a polynucleotide construct encoding ClndR7 and a truncated CD19 polypeptide (CD19t), with a T2A self-cleaving peptide sequence in between.

FIG. 5 depicts a FACS analysis for ClndR7-expressing T cells expressing the polynucleotide construct of FIG. 4. This analysis indicates that Her2tG can be used as a surface marker for ClndR7 expression, as it correlates with the co-expressed CD19t.

FIG. 6 depicts a FACS analysis of untreated (dark peak) or IL21-treated (light peak) CD8+ T cells for the presence of phosphorylated STAT3 (pSTAT3).

FIG. 7A depicts a FACTS analysis of unmodified (dark peak) or CindR-expressing (light peak) CD8+ T cells with ClndR7 expression for the presence of phosphorylated STAT5 (pSTAT3).

FIG. 7B depicts a FACTS analysis of unmodified (dark peak) or CindR-expressing (light peak) CD8+ T cells with ClndR7 expression for the presence of phosphorylated STAT3 (pSTAT3).

FIG. 7C depicts a FACS analysis of unmodified (dark peak) or CindR-expressing (light peak) CD8+ T cells with ClndR7/21 expression for the presence of phosphorylated STAT5 (pSTAT5).

FIG. 7D depicts a FACTS analysis of unmodified (dark peak) or CindR-expressing (light peak) CD8+ T cells with ClndR7/21 expression for the presence of phosphorylated STAT3 (pSTAT3).

FIG. 8A depicts a bar chart analysis of median fluorescent intensity (MFI) of cells (n=3 donors) subjected to intracellular flow cytometric quantification of phosphorylated STAT3 (pSTAT3). The T cells were engineered to express a non-active control protein (Her2tG) or one of the three ClndRs. Control cells were either untreated or pre-treated with IL-7 or IL-21. Cells were gated on lymphocytes, singlets, or dead stain excluding cells. Significant value markers are as determined through One-Way ANOVA, in which “*” indicates p<0.05, “**” indicates p<0.01, “***” indicates p<0.001, and “****” indicates p<0.0001.

FIG. 8B depicts a bar chart analysis of median fluorescent intensity (MFI) of cells (n=3 donors) subjected to intracellular flow cytometric quantification of phosphorylated STAT5 (pSTAT5). The T cells were engineered to express a non-active control protein (Her2tG) or one of the three ClndRs. Control cells were either untreated or pre-treated with IL-7 or IL-21. Cells were gated on lymphocytes, singlets, or dead stain excluding cells. Significant value markers are as determined through One-Way ANOVA, in which “*” indicates p<0.05, “**” indicates p<0.01, “,***” indicates p<0.001, and “****” indicates p<0.0001.

FIG. 9 depicts a schematic of a chimeric receptor ClndR7/21, with the flexible linker section (7), which was tested with alternative linker candidates GGG (SEQ. ID. NO: 23), GGGGS (SEQ. ID. NO: 24), (GGGGS)₂ (SEQ. ID. NO: 25) or (GGGGS)₃ (SEQ. ID NO: 26).

FIG. 10A depicts a bar chart analysis of median fluorescent intensity (MFI) of cells subjected to intracellular flow cytometric quantification of phosphorylated STATS (pSTAT5). Relative levels of MFI were relative to levels in mock cells. Measured levels includes those for, mock cells, mock cells with IL-21 treatment, ClndR7 cells, or ClndrR7/21 cells with one of the linker candidates GGG, GGGGS, (GGGGS)₂, or (GGGGS)₃.

FIG. 10B depicts a bar chart analysis of median fluorescent intensity (MF) of cells subjected to intracellular flow cytometric quantification of phosphorylated STAT3 (pSTAT3). Relative levels of MFI were relative to levels in mock cells. Measured levels includes those for, mock cells, mock cells with IL-21 treatment, ClndR7 cells, or ClndrR7/21 cells with one of the linker candidates GGG, GGGGS, (GGGGS)₂, or (GGGGS)₃.

FIG. 11A depicts an example schematie of a construct for introducing Her2tG into a T cell. This construct comprises Her2tG, MND, EF1a, CD19CAR, T2A, DHFRdm, P2A, and EGFRt sequences in a PBNP construct.

FIG. 11B depicts an example schematic of a construct for introducing ClndR7 into a T cell. This construct comprises ClndR7, MND, EF1a, CD19CAR, T2A, DHFRdm, P2A, and EGFRt sequences in a PBNP construct.

FIG. 11C depicts an example schematic of a construct for introducing ClndR7/21 into a T cell. This construct comprises ClndR7/21, MND, EF1a, CD19CAR, T2A, DHFRdm, P2A, and EGFRt sequences in a PBNP construct.

FIG. 11D depicts an example schematic of a construct for introducing ClndR21 into a T cell. This construct comprises ClndR21, MIND, EF1a, CD19CAR, T2A, DHFRdm, P2A, and EGFRt sequences in a PBNP construct.

FIG. 12A is a schematic for an assay wherein effector anti-CD19 CAR T cells containing various chimeric receptors were cocultured with target Raji cells (target cells). At days 3, 6, 9, and 12, additional Raji cells were added to the coculture. Tumor cell presence was monitored throughout the period.

FIG. 12B depicts a graph of the amount of red fluorescent signal per T cell group from Donor A. CAR T cells were subjected to repeated exposures to Raji tumor cells in vitro at three-day intervals (black arrows). Raji cells had been engineered to express the red fluorescent protein mCherry, and an Incucyte live cell fluorescent imager was used to monitor the presence of tumor cells over the course of three weeks.

FIG. 12C depicts a graph of the amount of red fluorescent signal per T cell group from Donor B. CAR T cells were subjected to repeated exposures to Raji tumor cells in vitro at three-day intervals (black arrows). Raji cells had been engineered to express the red fluorescent protein mCherry, and an Incucyte live cell fluorescent imager was used to monitor the presence of tumor cells over the course of three weeks.

FIG. 12D depicts a graph of the amount of red fluorescent signal per T cell group from Donor C. CAR T cells were subjected to repeated exposures to Raji tumor cells in vitro at three-day intervals (black arrows). Raji cells had been engineered to express the red fluorescent protein mCherry, and an Incucyte live cell fluorescent imager was used to monitor the presence of tumor cells over the course of three weeks.

FIG. 12E depict a graph for tumor signal over time for cocultures of Raji cells and anti-CD19 CAR T cells containing various chimeric receptors.

FIG. 12F depicts a graphs for tumor signal over time for cocultures of Raji cells and anti-CD19 CAR T cells containing control marker only receptors treated with IL-2 and/or IL-21.

FIG. 13A is a schematic for an assay in which effector anti-CD19 CAR T cells containing various chimeric receptors were cocultured with target Raji cells (target cells). At day 7, the co-culture was re-seeded. At days 7 and 14, the T cells were quantified.

FIG. 13B is a graph of relative fold expansion of CAR T cells containing various chimeric receptors, relative to expansion of CAR T cells containing a marker and treated with IL7 and IL21. Lines in the graph from top-most to lowest relate to: ClndR21, ClndR7/21, ClndR21, marker only.

FIG. 13C is a bar chart depicting the average relative fold expansion at day 14 across three donors with statistical significance indicated by asterisks. Statistical analysis was conducted using a one-way ANOVA test with these p-value annotations: p<0.05*, 0.0001****.

FIG. 14A is a schematic for an assay in which effector anti-CD19 CAR T cells containing various chimeric receptors were cocultured with target Raji cells (target cells). At day 7, exogenous cytokines were removed from the culture medium. An annexin V analysis was performed at day 10.

FIG. 14B is a bar chart depicting percentage of cells at day 10 that were: 7-AAD+ (dead cells), 7-AAD⁻ Annexin V⁻ (healthy cells), or 7-AAD⁻ Annexin V⁺ (pre-apoptotic cells). Statistical analysis was conducted using a one-way ANOVA test with these p-value annotations: p<0.05*, 0.01**.

FIG. 14C is a graph of relative fold change in live CAR T cells containing various chimeric receptors over time.

FIG. 15A is a schematic for an assay in which effector anti-CD19 CAR T cells containing various chimeric receptors were cocultured with target Raji cells (target cells). At day 3, the co-culture was re-seeded. At days 3 and 6, cell surface markers were analyzed by a flow cytometry.

FIG. 15B depicts graphs for percentage of CD8+ cells that were CD45RA+, CD62L+ and CCR7+ at day 3 (left panel) or at day 6 (right panel).

FIG. 16 depicts a graph of levels of flux in mice administered CD19+ tumor cells containing a fluorescent marker at day 0. At day 7, mice were administered anti-CD19 CAR cells which also contained various chimeric receptors. Lines in the graph from top-most to lowest (at day 15) relate to: mock; MND Her2tG CD19CAR; MIND ClndR7 CD19CAR; MIND ClndR7/21 CD19CAR; and MND ClndR21 CD19CAR.

### DETAILED DESCRIPTION

Successful adoptive T cell therapy requires a robust expansion and persistence of administered T cells, and the environmental signals received by the T cell contribute heavily to these behaviors. In preclinical models, chimeric antigen receptor (CAR) T cell therapy can be improved by supplementing the therapy with gamma chain cytokines, which are soluble factors that promote T cell growth and survival. However, systemic administration of cytokines to patients is not a therapeutically viable solution, as clinical trials have shown that such intervention leads to toxic side-effects (Jeught V. et al. (2014) Oncotarget 6:1359-81). In order to confer the benefits of cytokine supplementation to CAR T cell therapy without incurring systemic toxicity, a panel of chimeric cytokine receptors has been engineered that provides T cell-intrinsic constitutive interleukin signaling. Chimeric cytokine receptors recapitulated the signaling events of a specific gamma chain cytokine. Moreover, CAR T cells containing certain chimeric cytokine receptors had unexpectedly enhanced activities.

Chimeric cytokine receptor-expressing CAR T cells were subjected to serial tumor challenges in vitro and the capacity of T cell groups to survive, proliferate and eliminate tumor cells was examined. It was found that chimeric cytokine receptor-expressing CAR T cells exhibited increased proliferation and survival in the absence of exogenous cytokines.

Optimum T cell activation and expansion requires three signals- T cell receptor activation, co-stimulation and stimulatory cytokines. In CAR T cells, the CAR can provide the first two signals, but the third signal remains dependent on environmental/exogenous cytokines, which can be scarce in a tumor microenvironment. While cytokine supplementation can improve the efficacy of CAR T cell therapy, systemic cytokine administration has led to toxicities in clinical trials. Developing methods to selectively provide the third signal to CAR T cells without or reducing systemic toxicities is clinically valuable.

Disclosed herein are chimeric cytokine receptors comprising interleukin-7 domains and/or interleukin-21 domains for combinatorial interleukin signaling. In some alternatives, the cytokine receptor is ClndR7. In some alternatives, the cytokine receptor is ClndR7/21. In some alternatives, the cytokine receptor is ClndR21. In some alternatives, the hybrid cytokine receptor features signaling outputs of distinct endogenous receptors.

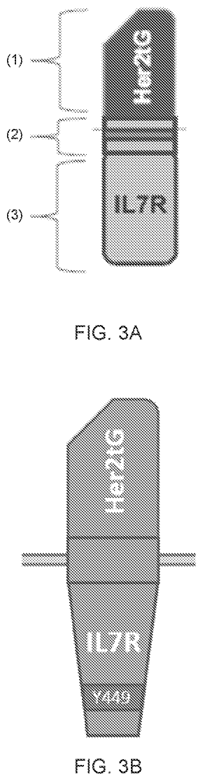

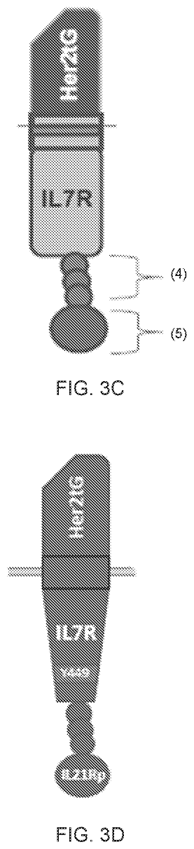

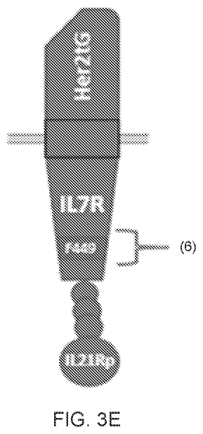

As disclosed herein, a cytokine-independent receptor for interleukin-7 and interleukin-21 signaling (ClndR7/21) has been designed, which is composed of fused elements of human interleukin-7 receptor (IL7R) and human interleukin-21 receptor (IL21R). Briefly, ClndR7/21 includes: (1) an extracellular domain of surface marker protein, a truncated HER2 polypeptide (Her2tG); (2) an IL7R transmembrane domain with an insertion to allow for receptor homodimerization and constitutive signaling; (3) an intracellular domain of IL7R; and (4) a peptide from the IL21R appended to the C-terminus of the IL7R via a flexible linker sequence (FIG. 3A).

Pre-clinical studies have shown the ability of cytokine supplementation to improve CAR T cell therapy. However, systemic cytokine administration is not a clinically viable option, as patients present with dose-limiting toxicities in clinical trials. The high-level goal of this invention is to confer the therapeutic benefits of cytokine supplementation to CAR T cells without incurring the systemic toxicities. Furthermore, by combining signaling outputs from two cytokines with potentially complementary effects on T cells, ClndR7/21 offers a novel approach to further optimize the signaling that drive CAR T cell expansion, persistence, and anti-tumor activity. IL7 increases T cell survival and proliferative activity, whileIL21 bolsters T cell cytotoxic capacity and resistance to exhaustion. The promotion of pro-therapeutic effects of both cytokines in CAR T cells with a single transgenic receptor in the form of ClndR7/21 is desired. The commercial application of ClndR7/21 is useful as a supplement to CAR T cell therapy or other types of adoptive T cell therapy.

As disclosed herein, ClndR7/21 offers the first constitutively active receptor with signaling outputs from both IL7 and IL21, which can further enable CAR T cells to be engineered with T cell intrinsic, constitutive IL7 and IL21 signaling, and promote T cell activity that leads to greater efficacy of CAR T cell therapy. These effects included increased CAR T cell expansion, persistence, cytotoxicity and resistance to T cell exhaustion. Preliminary studies in mice also indicated that ClndR7/21 and ClndR21 enhanced CAR T cell-mediated tumor suppression.

### Terms

Terms in the disclosure herein should be given their plain and ordinary meaning when read in light of the specification. One of skill in the art would understand the terms as used in view of the whole specification.

The term “comprising” as used herein is synonymous with “including,” “containing,” or “characterized by,” and is inclusive or open-ended and does not exclude additional, unrecited elements or method steps.

As used herein, “a” or “an” may mean one or more than one.

As used herein, the term “about” indicates that a value includes the inherent variation of error for the method being employed to determine a value, or the variation that exists among experiments.

As used herein, “chimeric receptor” can include a synthetically designed receptor comprising a ligand binding domain of an antibody or other protein sequence that binds to a molecule associated with a disease or disorder and is linked, preferably via a spacer domain, to one or more intracellular signaling, domains of a T cell or other receptors, such as a costimulatory domain. Chimeric receptors can also be referred to as artificial T cell receptors, chimeric T cell receptors, chimeric immunoreceptors, or chimeric antigen receptors (CARs).

As used herein, “chimeric cytokine receptor” can include a synthetically designed receptor comprising a cytokine tethered to an extracellular domain of a cytokine receptor polypeptide, a transmembrane domain, and an intracellular cytokine receptor domain linked to the extracellular domain of a cytokine receptor polypeptide via the transmembrane domain. In some embodiments, the cytokine can be selected from a type I cytokine receptor, such as IL-2, IL-4, IL-7, IL-9, IL-13, IL, 15, or IL-21. In some embodiments, the extracellular domain of a cytokine receptor polypeptide can be derived from a type I cytokine receptor, such as IL-2, IL-4, IL-7, IL-9, IL-13, IL, 15, or IL-21. In some embodiments, the transmnembrane domain can be derived from a type I cytokine receptor, such as IL-2, IL-4, IL-7, IL-9, IL-13, IL, 15, or IL-21. In some embodiments, the intracellular domain of a cytokine receptor polypeptide can be derived from a type I cytokine receptor, such as IL-2, IL-4, IL-7, IL-9, IL-13, IL, 15, or IL-21.

As used herein, “chimeric antigen receptor” (CAR), also known as chimeric T-cell receptors, can refer to artificial T-cell receptors that are engineered receptors, which graft an arbitrary specificity onto an immune effector cell. These receptors can be used to graft the specificity of a monoclonal antibody onto a T-cell, for example; with transfer of their coding sequence facilitated by retroviral vectors, or any other suitable gene delivery system. The structure of the CAR can comprise single-chain variable fragments (scFv) derived from monoclonal antibodies, fused to CD3-zeta transmembrane and endodomain. Such molecules result in the transmission of a zeta signal in response to recognition by the scFv of its target. Some alternatives utilize a gene delivery vector having a self-inactivating transposase system. In some alternatives, the gene delivery vector further comprises a sequence for at least one protein. In some alternatives, the protein is a chimeric antigen receptor. Chimeric receptors can also be referred to as artificial T cell receptors, chimeric T cell receptors, chimeric immunoreceptors, and/or CARs. These CARs are engineered receptors that can graft an arbitrary specificity onto an immune receptor cell. CARs may include the antibody or antibody fragment, spacer, signaling domain, and/or transmembrane region. However, due to the surprising effects of modifying the different components or domains of the CAR, such as the epitope binding region (for example, antibody fragment, scFv, or portion thereof), spacer, transmembrane domain, and/or signaling domain), the components of the CAR are described herein in some contexts to include these features as independent elements. The variation of the different elements of the CAR can, for example, lead to stronger binding affinity for a specific epitope.

Artificial T-cell receptors, or CARs, can be used as a therapy for cancer or viral infection using a technique called adoptive cell transfer. T-cells are removed from a subject and modified so that they express receptors specific for a molecule displayed on a cancer cell or virus, or virus-infected cell. The genetically engineered T-cells, which can then recognize and kill the cancer cells or the virus infected cells or promote clearance of the virus, are reintroduced into the subject. In some alternatives, the gene delivery vector can comprise a sequence for a chimeric antigen receptor. In some alternatives, a method of generating engineered multiplexed T-cells for adoptive T-cell immunotherapy is provided. In the broadest sense the method can comprise providing the gene delivery vector of any one of the alternatives described herein, introducing the gene delivery vector into a T-cell, and selecting the cells comprising the gene delivery vector, wherein selecting comprises isolating the T-cells expressing a phenotype under selective pressure.

T-cell co-stimulation is desired for development of an effective immune response and this event occurs during the activation of lymphocytes. A co-stimulatory signal is antigen non-specific and is provided by the interaction between co-stimulatory molecules expressed on the membrane of the antigen bearing cell and the T-cell. Co-stimulatory molecules can include but are not limited to CD28, CD80, or CD86. In some alternatives, a method for generating engineered multiplexed T-cells for adoptive T-cell immunotherapy is provided. In some alternatives, the T-cell is a chimeric antigen receptor bearing T-cell. In some alternatives, the chimeric antigen receptor bearing T-cell is engineered to express co-stimulatory ligands. In some alternatives, methods are provided for treating, inhibiting, or ameliorating cancer or a viral infection in a subject. In the broadest sense, the method can comprise administering to the subject a T-cell of any of the alternatives described herein. In some of these alternatives, the subject is an animal, such as domestic livestock or a companion animal and in other alternatives, the subject is a human. In some of these alternatives, the chimeric antigen bearing T-cell is engineered to express a co-stimulatory molecule. In some alternatives, the gene delivery vector comprises a sequence for at least one co-stimulatory molecule. In some alternatives, the gene delivery vector is at least 1 kB to 20 kB.

As described herein, “genetically modify” and “genetically modified” can include a process for modifying an organism or a cell such as a bacterium, T-cell, bacterial cell, eukaryotic cell, insect, plant or mammal with genetic material, such as nucleic acid, that has been altered using genetic engineering techniques. For example, a nucleic acid such as DNA can be inserted in the host genome by first isolating and copying the genetic material of interest using molecular cloning methods to generate a DNA sequence, or by synthesizing the DNA, and then inserting this construct into the host organism. Genes can also be removed, or “knocked out”, using a nuclease. Gene targeting is a different technique that uses homologous recombination to change an endogenous gene, and can be used to delete a gene, remove exons, add a gene, or introduce point mutations.

Genetic modification performed by transduction is described herein. “Transduction” refers to methods of transferring genetic material, such as, for example, DNA or RNA, to a cell by way of a vector. Common techniques use viral vectors, electroporation, and chemical reagents to increase cell permeability. The DNA can be transferred by a virus, or by a viral vector. Described herein are methods for modifying immune CD4+ and/or CD8+ T-cells. In order to achieve high expression of therapeutic genes and/or to increase the amount of chimeric antigen receptors on a cell surface, for example, T-cells are transduced with genetic material encoding a protein or a chimeric antigen receptor. T-cells can be genetically modified using a virus, for example. Viruses commonly used for gene therapy are adenovirus, adeno-associated virus (AAV), retroviruses or lentiviruses, for example.

Various transduction techniques have been developed, which utilize recombinant infectious virus particles for delivery of the nucleic acid encoding a chimeric antigen receptor. This represents a currently preferred approach to the transduction of T lymphocytes. As described herein, the viral vectors used for transduction can include virus vectors derived from simian virus 40, adenoviruses, AAV, lentiviral vectors, or retroviruses, Thus, gene transfer and expression methods are numerous but essentially function to introduce and express genetic material in mammalian cells. Several of the above techniques can be used to transduce hematopoietic or lymphoid cells, including calcium phosphate transfection, protoplast fusion, electroporation, or infection with recombinant adenovirus, adeno-associated virus, lentivirus, or retrovirus vectors. Primary T lymphocytes have been successfully transduced by electroporation and by retroviral or lentiviral infection. As such, retroviral and lentiviral vectors provide a highly efficient approach for gene transfer to eukaryotic cells, such as T-cells. Moreover, retroviral or lentiviral integration takes place in a controlled fashion and results in the stable integration of one or a few copies of the new genetic information per cell. As described herein, the cells can be transduced in situ.

As used herein, a “vector” or “construct” can include a nucleic acid used to introduce heterologous nucleic acids into a cell that can also have regulatory elements to provide expression of the heterologous nucleic acids in the cell. Vectors include but are not limited to plasmid, minicircles, yeast, or viral genomes. In some embodiments, the vectors are plasmid, minicircles, viral vectors, DNA or mRNA. In some embodiments, the vector is a lentiviral vector or a retroviral vector. In some embodiments, the vector is a lentiviral vector. As used herein, “Vpx” can include a virion associated protein that is encoded by HIV type 2 and in some simian immunodeficiency virus strains. Vpx can enhance HIV-2 replication in humans. Lentiviral vectors packaged with Vpx protein can led to an increase in the infection of myeloid cells, when used in transfections. In some embodiments, the lentiviral vector is packaged with a Vpx protein. As used herein, “Vpr” protein can refer to Viral Protein R, which is a 14 kDa protein, which plays an important role in regulating nuclear import of the I-IV-1 pre-integration complex and is required for virus replication in non-dividing cells. Non-dividing cells can include macrophages, for example. In some embodiments, the lentiviral vector can be packaged with a Vpr protein, or a Vpr protein portion thereof. In some embodiments, the lentiviral vector is packaged with a viral accessory protein. In some embodiments, the viral accessory protein is selected from the group consisting of Vif, Vpx, Vpu, Nef and Vpr. These accessory proteins such as, for example vif, Vpx, vpu or nef interact with cellular ligands to act as an adapter molecule to redirect the normal function of host factors for virus-specific purposes. HIV accessory proteins are described in Strebel et al. (“HIV Accessory Proteins versus Host Restriction Factors, Curr Opin Virol. 2013 December; 3(6): 10.1016/j.coviro.2013.08.004; hereby expressly incorporated by reference in its entirety).

As used herein, a “promoter” can include a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription are often characterized by consensus nucleotide sequences. Without being limiting, these promoter elements can include RNA polymerase binding sites, TATA sequences, CAAT sequences, or differentiation-specific elements (DSEs; McGehee et al., Mol. Endocrinol. 7:551 (1993); hereby expressly incorporated by reference in its entirety), cyclic AMP response elements (CREs), serum response elements (SREs; Treisman et al., Seminars in Cancer Biol. 1:47 (1990); hereby expressly incorporated by reference in its entirety), glucocorticoid response elements (GREs), or binding sites for other transcription factors, such as CRE/ATF (O'Reilly et al., J. Biol. Chem. 267:19938 (1992); hereby expressly incorporated by reference in its entirety), AP2 (Ye et al., J. Biol. Chem. 269:25728 (1994); hereby expressly incorporated by reference in its entirety), SP1, cAMP response element binding protein (CREB; Loeken et al, Gene Expr. 3:253 (1993); hereby expressly incorporated by reference in its entirety) or octamer factors (see, in general, Watson et al., eds., Molecular Biology of the Gene, 4th ed. (The Benjamin/Cummings Publishing Company, Inc. 1987; hereby expressly incorporated by reference in its entirety)), and Lemaigre and Rousseau, Biochem. J. 303:1 (1994); hereby expressly incorporated by reference in its entirety). As used herein, a promoter can be constitutively active, repressible or inducible. If a promoter is an inducible promoter, then the rate of transcription increases in response to an inducing agent. In contrast, the rate of transcription is not regulated by an inducing agent if the promoter is a constitutive promoter. Repressible promoters are also known.

As used herein, “treat,” “treating,” “treated,” or “treatment” can refer to both therapeutic treatment and prophylactic or preventative treatment depending on the context.

As used herein, “ameliorate,” “ameliorating,” “amelioration,” or “ameliorated” in reference to a disorder can refer to reducing the symptoms of the disorder, causing stable disease, or preventing progression of the disorder. For disorders such as cancer, this can include reducing the size of a tumor, reducing cancer cell growth or proliferation or cancer cell number, completely or partially removing the tumor (e.g., a complete or partial response), causing stable disease, preventing progression of the cancer (e.g., progression free survival), or any other effect on the cancer that would be considered by a physician to be a therapeutic.

As used herein, “administer,” administering,” or “administered” can refer to methods of introducing the compound, or pharmaceutically acceptable salt thereof, or modified cell composition, to a patient or subject, including, but not limited to, oral, intravenous, intramuscular, subcutaneous, or transdermal.

As used herein, “subject” or “patient,” can refer to any organism upon which the embodiments described herein may be used or administered, e.g., for experimental, diagnostic, prophylactic, and/or therapeutic purposes. Subjects or patients include, for example, animals. In some embodiments, the subject is mice, rats, rabbits, non-human primates, or humans. In some embodiments, the subject is a cow, sheep, pig, horse, dog, cat, primate or a human.

As used herein, “co-administration” can refer to the administration of more than one therapeutic agent in combination with another. Each reagent can be administered sequentially or concurrently, such that each reagent can be in the blood stream of an organism at the same time.

### Certain Cytokine Receptors

Some embodiments provided herein include a chimeric cytokine receptor polypeptide. In some embodiments, chimeric cytokine receptor comprises (i) an extracellular domain, such as a cell surface selectable polypeptide, such as an extracellular HER2 domain, an extracellular EGFR domain, or an extracellular CD19 domain; (ii) a transmembrane interleukin-7 receptor (IL-7R) domain; and (iii) an intracellular IL-7R domain. In some embodiments, the extracellular domain is a domain selected such that cells expressing the domain can be readily identified and/or selected.

In some embodiments, the transmembrane IL-7R domain is modified to promote dimerization of the chimeric receptor, and/or constitutive signaling. In some embodiments, the chimeric cytokine receptor further comprises a GMCSF signal sequence. In some embodiments, the GMCSF signal sequence has at least 80% sequence identity to the amino acid sequence of SEQ ID NO 7, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 7. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, the extracellular domain comprises the extracellular HER2 domain. In some embodiments, the extracellular domain comprises the extracellular CD19 domain comprising a truncated CD19 polypeptide (CD19t). In some embodiments, the CD19t comprises an amino acid sequence having at least 80%, 85%, 90%, 91%, 92%, 93%, 94%, 95%, 96%, 97%, 98%, 99% or is 100% sequence identity to the amino acid sequence of SEQ ID NO:28. In some embodiments, the extracellular domain comprises the extracellular EGER domain, such as a truncated EGFR polypeptide (EGFRt).

In some embodiments, the extracellular HER2 domain comprises a polypeptide having at least 80% sequence identity to the amino acid sequence of SEQ ID NO: 8, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 8. In some such embodiments, the percentage identity is greater than 80%, 85%, 90%, 91%, 92%, 93%, 94%, 95%, 96/0, 97/), 98%, 99% or is 100% or is within a range defined by any two of the aforementioned percentages. In some such embodiments, the percentage identity is greater than about 95%. In some such embodiments, the percentage identity is greater than 95%.

In some embodiments, the extracellular HER2 domain lacks domain I, domain II, and domain III of an extracellular HER2 protein. In some embodiments, the transmembrane IL-7R domain has at least 80% sequence identity to the amino acid sequence of SEQ ID NO: 9, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 9. In some such embodiments, the percentage identity is greater than 80%, 85%, 900, 91%, 92% 93, 94%, 95%, 96%, 97%, 98% 99% or is 100% or is within a range defined by any two of the aforementioned percentages. In some such embodiments, the percentage identity is greater than about 95%. In some such embodiments, the percentage identity is greater than 95%.

In some embodiments, the transmembrane IL-7R domain is modified to promote dimerization of the chimeric receptor and comprises the amino acid sequence TCP. In some embodiments, the transmembrane IL-7R domain has at least 80% sequence identity to the amino acid sequence of SEQ ID NO: 10, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 10. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, the chimeric cytokine receptor further comprises an interleukin-21 receptor (IL-21R) polypeptide attached to the C-terminus of the IL-7R domain via a linker. In some embodiments, the linker has at least 80% sequence identity to the amino acid sequence of SEQ ID NO: 11, such as at least 80%, 81%, 82%, 83/a, 84%, 85%, 86%, 87%, 88%, 89%, 90%, 91%, 92%, 930, 940, 95%, 96%, 97%, 98%, or 100% sequence identity to the amino acid sequence of SEQ ID NO: 11. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%. In some embodiments, the linker is selected from the group consisting of SEQ ID NO: 23, SEQ ID NO: 24, SEQ ID NO: 25, and SEQ ID NO: 26. In some such embodiments, linker is selected from a sequence with a percentage identity that is greater than 80%, 85%, 90%, 91%, 92%, 93%, 94%, 95%, 96%, 97%, 98%, 99% or is 100% or is within a range defined by any two of the aforementioned percentages, to any one of SEQ ID NO: 23, SEQ ID NO: 24, SEQ ID NO: 25,and SEQ ID NO: 26. In some such embodiments, the percentage identity is greater than about 95% to any one of SEQ ID NO: 23, SEQ ID NO: 24, SEQ ID NO: 25,and SEQ ID NO: 26. In some such embodiments, the percentage identity is greater than 95% to any one of SEQ ID NO: 23, SEQ ID NO: 24, SEQ ID NO: 25,and SEQ ID NO: 26. In some embodiments, the linker has a length from about 3 amino acids to about 30 amino acids. In some embodiments, the linker has a length of about 3 amino acids. In some embodiments, the linker has a length of about 4 amino acids. In some embodiments, the linker has a length of about 5 amino acids. In some embodiments, the linker has a length of about 10 amino acids. In some embodiments, the linker has a length of about 15 amino acids. In some embodiments, the linker has a length of about 20 amino acids. In some embodiments, the linker has a length of about 30 amino acids.

In some embodiments, the IL-21R-peptide has at least 80% sequence identity to the amino acid sequence of SEQ ID NO: 12, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 12. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, the intracellular IL-7R domain is modified to ablate a STAT5 signaling motif. In some embodiments, the intracellular IL-7R domain has at least 80% identity to the amino acid sequence of SEQ ID NO: 13, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 13. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, the intracellular IL-7R domain comprises a Y449E mutation. In some embodiments, the chimeric cytokine receptor comprises an amino acid sequence having at least 80% sequence identity to any one of SEQ ID NO: 1, SEQ ID NO: 2, or SEQ ID NO: 3, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO: 1, SEQ ID NO: 2, or SEQ ID NO: 3. In some such embodiments, the percentage identity is greater than 80%, 85%, 90%, 91%, 92%, 93%, 94%, 95%, 96%, 97%, 98%, 99%4 or is 100% or is within a range defined by any two of the aforementioned percentages to any one of SEQ ID NO: 1, SEQ ID NO: 2, or SEQ ID NO: 3. In some such embodiments, the percentage identity is greater than about 95% to any one of SEQ ID NO: 1, SEQ ID NO: 2, or SEQ ID NO: 3. In some such embodiments, the percentage identity is greater than 95% to any one of SEQ ID NO: 1, SEQ ID NO: 2, or SEQ ID NO: 3.

### Certain Polynucleotides

Some embodiments provided herein include a polynucleotide encoding a chimeric cytokine receptor polypeptide. In some embodiments, the polynucleotide encodes the chimeric cytokine receptor of any of the embodiments disclosed herein. In some embodiments, the polynucleotide preferably comprises a nucleotide sequence having at least 80% sequence identity to any one of SEQ ID NO: 4, SEQ ID NO: 5, or SEQ ID NO: 6, such as at least 80%, 81%, 82%, 83%, 84%, 85%, 86%, 87%, 88%, 89%, 90%, 91% 92%, 93%, 94%, 95%, 96%, 97%, 98%, 99%, or 100% sequence identity to any one of SEQ ID NO: 4, SEQ ID NO: 5, or SEQ ID NO: 6. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages to any one of SEQ ID NO: 4, SEQ ID NO: 5, or SEQ ID NO: 6. In some such embodiments, the percentage identity is greater than about 95% to any one of SEQ ID NO: 4, SEQ ID NO: 5, or SEQ ID NO: 6. In some such embodiments, the percentage identity is greater than 95% to any one of SEQ ID NO: 4, SEQ ID NO: 5, or SEQ ID NO: 6.

In some embodiments, the polynucleotide further comprises a promoter. In some embodiments, the promoter is an inducible promoter or a constitute promoter. In some embodiments, the promoter is an EF1alpha promoter, or an MND promoter (e.g., SEQ ID NO: 21 or a promoter having at least 80% sequence identity to SEQ. ID. NO: 21, such as at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 21). In some such embodiments, the percentage identity to SEQ, ID. NO:21 is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%4. In some such embodiments, the percentage identity is greater than 95%.

In some embodiments, the polynucleotide further comprises a transcriptional terminator. In some embodiments, the transcriptional terminator is an RBG PolyA sequence (e.g., SEQ ID NO: 22 or a terminator having at least 80% sequence identity to SEQ. ID. NO: 22, such as at least 80%, 81%, 82%, 83%, 84%, 85%, 86%, 87%, 88%, 89%, 90%, 91′%, 92%, 93%, 94%, 95%, 96%, 97%, 98%, 99%, or 100% sequence identity to SEQ ID NO: 22). In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, the polynucleotide further comprises a translational factor. In some embodiments, the translational factor is EF1a.

In some embodiments, the polynucleotide further comprises a nucleic acid encoding a selectable marker, such as a truncated CD19 (CD19t) polypeptide, or a truncated EGFR (EGFRt) polypeptide. In some embodiments, the nucleic acid encodes a truncated CD19 (CD19t), such as SEQ ID NO: 30 or a the nucleic acid encodes a CD19t having at least 80% sequence identity to SEQ. ID. NO: 30, such as at least 80%, 81%, 82%, 83%, 84%, 85%, 86%, 87%, 88%, 89%, 90%, 91%, 92%, 93%, 94%, 950, 96%, 97%, 98%, 99%, or 100% sequence identity to SEQ ID NO: 30. In some such embodiments, the percentage identity is greater than 80%, 85%, 90%, 91%, 92%, 93%, 94%, 95%, 96%, 97%, 98%, 99% or is 100% or is within a range defined by any two of the aforementioned percentages. In some such embodiments, the percentage identity is greater than about 95%. In some such embodiments, the percentage identity is greater than 95%.

In some embodiments, the polynucleotide further comprises a selection factor and/or resistance cassette. In some embodiments, the selection factor and/or resistance cassette encodes a double mutant dihydrofolate reductase (DHFRdm) polypeptide.

In some embodiments, the polynucleotide further comprises a ribosomal skip sequence and/or a sequence encoding a self-cleaving peptide. In some embodiments, the ribosomal skip sequence comprises a T2A skip sequence, and/or the self-cleaving peptide comprises a P2A self-cleaving peptide sequence.

In some embodiments, the polynucleotide further comprises a nucleic acid sequence encoding a chimeric antigen receptor, such as an anti-CD19 CAR.

### Vectors

Some embodiments disclosed herein concern a vector. In some embodiments, the vector comprises a polynucleotide described herein. In some embodiments, the vector is suitable for or configured for transduction into a cell.

In some embodiments, the vector comprises a viral vector. In some embodiments, the vector is selected from a lentiviral vector, an adeno-associated viral vector, or an adenoviral vector. In some embodiments, the vector comprises a lentiviral vector.

In some embodiments, a vector can include a polynucleotide provided herein encoding a chimeric cytokine receptor, and/or a polynucleotide encoding a CAR.

### Cells

Some embodiments disclosed herein concern a cell. In some embodiments, the cell comprises one or more of the polypeptide of any of the present embodiments, the polynucleotide of any of the present embodiments, and/or the vector of any of the present embodiments disclosed herein. In some embodiments, the cell comprises a polypeptide described herein. In some embodiments, the cell comprises a polynucleotide described herein. In some embodiment, the cell comprises a vector described herein. In some embodiments, the cell further comprises a polynucleotide encoding a chimeric antigen receptor (CAR), and/or a CAR protein. In some embodiments, the cell is a white blood cell (leukocyte). In some embodiments, the cell is a B cell. In some embodiments, the cell is a T cell. In some embodiments, the cell is a CD8+ T cell or a CD4+ T cell. In some embodiments, the cell is a CD8+ T cell. In some embodiments, the cell is a CD4+ T cell. In some embodiments, the cell is derived from a CD8+ T cell or a CD4+ T cell. In some embodiments, the cell is derived from a CD8+ T cell. In some embodiments, the cell is derived from a CD4+ T cell. In some embodiments, the cell is a primary cell. In some embodiments, the cell is a cell line. In some embodiments, the cell is produced from a cell culture. In some embodiments, the cell is taken directly from the tissue of a subject. In some embodiments, the cell is mammalian. In some embodiments, the cell is human. In some embodiments, the cell is ex vivo.

### Therapeutic Methods

Some embodiments concern a therapeutic method. In some embodiments, the method concerns the treating, inhibiting, or ameliorating a disease or disorder in a subject, such as a cancer. Some such methods include administering a cell provided herein to a subject in need thereof. In sonic embodiments, the method comprises administering the cell of any one of the embodiments disclosed herein to the subject in need thereof. In some embodiments, the cell includes chimeric cytokine receptor or a polynucleotide encoding the chimeric cytokine receptor. In some embodiments, the cell further comprises a CAR or a polynucleotide encoding the CAR.

In some embodiments, the disease or disorder is an autoimmune disease. In some embodiments, the disease or disorder is inflammation. In some embodiments, the disease or disorder is a cancer. In some embodiments, the cancer can include a solid tumor such as a colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma. In some embodiments, the cancer comprises a brain cancer.

In some embodiments, the treatment or amelioration of the disease or disorder lacks co-administration of a cytokine to the subject. In some embodiments, the method further comprises co-administering a cytokine to the subject, wherein the dose of the cytokine administered is reduced compared to the dose of the cytokine co-administered to a subject who has been administered a cell comprising a CAR, which lacks the polypeptide of any one of the present embodiments, the polynucleotide of any one of the present embodiments, and/or the vector of any one of the present embodiments disclosed herein.

Some of the chimeric cytokine receptors provided herein can reduce or eliminate a need to supplement a therapy to a subject by co-administering a cytokine. For example, administration of a CAR T cell to a subject, which does not contain a chimeric cytokine receptor can include co-administration of an exogenous cytokine to further stimulate or activate the CAR T cells that have been administered to the subject. As described herein, T cells containing a chimeric cytokine receptor provided herein can be provided in a sufficiently stimulated and/or activated state in the absence of an exogenous cytokine, or a substantially reduced dose of an exogenous cytokine compared to a T cell not containing a chimeric cytokine receptor provided herein. In some embodiments, the treatment or amelioration of the cancer lacks co-administration of a cytokine to the subject. Some embodiments can also include co-administering a cytokine to the subject, wherein the dose of the cytokine is reduced compared to the dose of the cytokine co-administered to a subject who has been administered a cell comprising a CAR, which lacks a chimeric cytokine receptor provided herein. In some such embodiments, the dose of a cytokine co-administered to a subject who has been administered a cell comprising a CAR and a chimeric cytokine receptor can be reduced in comparison to the dose of a cytokine co-administered to a subject who has been administered a cell comprising a CAR, which lacks a chimeric cytokine receptor by more than or more than about 10%, 2⁰%, 30%, 40%, 50%, 60%, 70%, 80%, 90%, 100%, or an amount that is within a range defined by any two of the aforementioned percentages.

In some embodiments, administration of a CAR T cell containing a chimeric cytokine receptor provided herein improves the removal of tumor from a subject compared to the removal of a tumor in a subject administered a CAR T cell lacking a chimeric cytokine receptor provided herein.

In some such embodiments, administration of a CAR T cell containing a chimeric cytokine receptor provided herein reduces the volume of a of tumor in a subject compared to the volume of a tumor in a subject who has been administered a CAR T cell lacking a chimeric cytokine receptor provided herein. In some such embodiments, the volume of a of tumor in a subject administration of a CAR T cell containing a chimeric cytokine receptor provided herein compared to the volume of a tumor in a subject who has been administered a CART cell lacking a chimeric cytokine receptor provided herein can be reduced by more than or more than about 10%, 20%, 30%, 40%, 50%, 60%, 70%, 80%, 90%, 100%, or an amount that is within a range defined by any two of the aforementioned percentages.

In some such embodiments, administration of a CAR T cell containing a chimeric cytokine receptor provided herein reduces the volume of a of tumor in a subject at a greater rate that a reduction in the volume of a tumor in a subject who has been administered a CAR T cell lacking a chimeric cytokine receptor provided herein.

In some embodiments, administration of a CAR T cell containing a chimeric cytokine receptor provided herein increases overall survival of a subject compared to the overall survival of a subject administered a CAR T cell lacking a chimeric cytokine receptor provided herein.

In some embodiments, the cytokine is selected from IL-7 or IL-21. In some embodiments, the cytokine comprises IL-7. In some embodiments, the cytokine comprises IL-21. In some embodiments, the cell is autologous to the subject. In some embodiments, the subject is mammalian. In some embodiments, the subject is human.

### Methods of Preparing Populations of Cells

Some embodiments provided herein concern methods of preparing a population of cells comprising a chimeric antigen receptor (CAR). In some embodiments, the method comprises (a) transducing a T cell with the polynucleotide of any one of the present embodiments, (b) transducing the T cell with a polynucleotide encoding a CAR, and (c) culturing the transduced T cell under conditions to stimulate activation and expansion of the T cell, wherein the culture media lacks an exogenous cytokine. In some embodiments, step (b) is performed before step (a). In some embodiments, step (a) and (b) are performed concurrently. In some embodiments, the cytokine is selected from IL-7 or IL-21. In some embodiments, the cytokine comprises IL-7. In some embodiments, the cytokine comprises IL-21. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian. In some embodiments, the cell is human. In some embodiments, the cell is ex vivo.

In some embodiments, the method comprises (a) transducing a T cell with the polynucleotide of any one of the present embodiments, (b) transducing the T cell with a polynucleotide encoding a CAR, and (c) culturing the transduced T cell under conditions sufficient to stimulate activation and expansion of the T cell, wherein the culture media comprises a reduced amount of an exogenous cytokine as compared to an amount sufficient to stimulate activation and expansion of the T cell lacking the polynucleotide encoding a chimeric receptor. In some embodiments, step (b) is performed before step (a). In some embodiments, step (a) and (b) are performed concurrently. In some embodiments, the cytokine is selected from IL-7 or IL-21. In some embodiments, the cytokine comprises IL-7, In some embodiments, the cytokine comprises IL-21. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian. In some embodiments, the cell is human. In some embodiments, the cell is ex vivo.

In some embodiments, the amount of an exogenous cytokine sufficient to stimulate activation and expansion of a T cell comprising a CAR and a chimeric cytokine receptor provided herein is reduced in comparison to the amount of an exogenous cytokine sufficient to stimulate activation and expansion of a T cell comparing a CAR and lacking a chimeric cytokine receptor provided herein by more than or more than about 10%, 20%, 30%, 40%, 50%, 60%, 70%, 80%, 90%, 100%, or by an amount that is within a range defined by any two of the aforementioned percentages. In some embodiments, the cytokine is selected from IL-7 or IL-21. In some embodiments, the cytokine comprises IL-21. In some embodiments, the cytokine comprises IL-7. In some embodiments, the cell is a CD8+ T cell or a CD4+ T cell. In some embodiments, the cell is derived from a CD8+ T cell or a CD4+ T cell. In some embodiments, the cell is a primary cell. In some embodiments, the cell is mammalian, such as human. In some embodiments, the cell is ex vivo.

Certain sequences useful with embodiments of the methods and compositions provided herein are listed in the following TABLE 1.

TABLE 1

| Feature | |
|---|---|
| (SEQ ID NO) | Sequence |
| CIndR7 | MLLLVTSLLLCELPHPAFLLIPCHPECQPQNGSVTCFGPEADQCVACAHY |
| (SEQ ID | KDPPFCVARCPSGVKPDLSYMPIWKFPDEEGACQPCPINCTHSCVDLDDK |
| NO: 1) | GCPAEQRASPLTGGGSGGGSPILLTCPTISILSFFSVALLVILACVLWKKRI |
| | KPIVWPSLPDHKKTLEHLCKKPRKNLNVSFNPESFLDCQIHRVDDIQARD |
| | EVEGFLQDTFPQQLEESEKQRLGGDVQSPNCPSEDVVITPESFGRDSSLTC |
| | LAGNVSACDAPILSSSRSLDCRESGKNGPHVYQDLLLSLGTINSTLPPPFS |
| | LQSGILTLNPVAQGQPILTSLGSNQEEAYVTMSSFYQNQ |
| CIndR7/21 | MLLLVTSLLLCELPHPAFLLIPCHPECQPQNGSVTCFGPEADQCVACAHY |
| (SEQ ID | KDPPFCVARCPSGVKPDLSYMPIWKFPDEEGACQPCPINCTHSCVDLDDK |
| NO: 2) | GCPAEQRASPLTGGGSGGGSPILLTCPTISILSFFSVALLVILACVLWKKRI |
| | KPIVWPSLPDHKKTLEHLCKKPRKNLNVSFNPESFLDCQIHRVDDIQARD |
| | EVEGFLQDTFPQQLEESEKQRLGGDVQSPNCPSEDVVITPESFGRDSSLTC |
| | LAGNVSACDAPILSSSRSLDCRESGKNGPHVYQDLLLSLGTTNSTLPPPFS |
| | LQSGILTLNPVAQGQPILTSLGSNQEEAYVTMSSFYQNQGGGGSGGGGSS |
| | PVECDFTSPGDEGPPRSYLRQWVVIPPPLSSPGPQAS |
| CIndR21 | MLLLVTSLLLCELPHPAFLLIPCHPECQPQNGSVTCFGPEADQCVACAHY |
| (SEQ ID | KDPPFCVARCPSGVKPDLSYMPIWKFPDEEGACQPCPINCTHSCVDLDDK |
| NO: 3) | GCPAEQRASPLTGGGSGGGSPILLTCPTISILSFFSVALLVILACVLWKKRI |
| | KPIVWPSLPDHKKTLEHLCKKPRKNLNVSFNPESFLDCQIHRVDDIQARD |
| | EVEGFLQDTFPQQLEESEKQRLGGDVQSPNCPSEDVVITPESFGRDSSLTC |
| | LAGNVSACDAPILSSSRSLDCRESGKNGPHVYQDLLLSLGTTNSTLPPPFS |
| | LQSGILTLNPVAQGQPILTSLGSNQEEAFVTMSSFYQNQGGGGSGGGGSS |
| | PVECDFTSPGDEGPPRSYLRQWVVIPPPLSSPGPQAS |
| CIndR7 | ATGCTGCTGCTGGTCACATCTCTGCTGCTGTGCGAGCTGCCCCATCCT |
| (SEQ ID | GCCTTTCTGCTGATCCCTTGTCACCCCGAGTGCCAGCCTCAGAATGGC |
| NO: 4) | AGCGTGACATGCTTTGGCCCTGAGGCCGATCAGTGTGTGGCCTGCGCT |
| | CACTACAAGGACCCTCCATTCTGCGTGGCCAGATGTCCTTCTGGCGTG |
| | AAGCCCGACCTGAGCTACATGCCCATCTGGAAGTTCCCCGATGAGGA |
| | AGGCGCCTGCCAGCCTTGTCCTATCAATTGCACACACAGCTGCGTGGA |
| | CCTGGACGACAAAGGATGTCCTGCCGAGCAGAGAGCCTCTCCACTTA |
| | CTGGCGGAGGAAGCGGCGGAGGATCTCCTATCCTGCTGACCTGTCCTA |
| | CAATCAGCATCCTGAGCTTTTTCAGCGTGGCCCTGCTCGTGATCCTGG |
| | CCTGTGTGCTGTGGAAGAAGCGGATCAAGCCCATCGTGTGGCCCAGC |
| | CTGCCTGACCACAAGAAAACCCTGGAACACCTGTGCAAGAAGCCCCG |
| | GAAGAACCTGAACGTGTCCTTCAATCCCGAGAGCTTCCTGGACTGCCA |
| | GATCCACAGAGTGGACGACATCCAGGCCAGAGATGAGGTGGAAGGCT |
| | TTCTGCAGGACACATTCCCTCAGCAGCTGGAAGAGAGCGAGAAGCAG |
| | AGACTCGGCGGAGATGTGCAGAGCCCTAATTGCCCTAGCGAGGACGT |
| | GGTCATCACCCCTGAGAGCTTCGGCAGAGATAGCAGCCTGACATGTCT |
| | GGCCGGCAATGTGTCCGCCTGTGATGCCCCTATCCTGAGCAGCTCCAG |
| | AAGCCTGGATTGCAGAGAGAGCGGCAAGAACGGCCCTCACGTGTACC |
| | AGGATCTGCTCCTGTCTCTGGGCACCACCAATAGCACACTGCCTCCAC |
| | CATTCAGCCTGCAGAGCGGCATCCTGACACTGAACCCTGTTGCTCAGG |
| | GACAGCCCATCCTGACCAGCCTGGGCAGCAATCAAGAAGAGGCCTAC |
| | GTCACCATGAGCAGCTTCTACCAGAACCAG |
| CIndR7/21 | ATGCTGCTGCTGGTCACATCTCTGCTGCTGTGCGAGCTGCCCCATCCT |
| (SEQ ID | GCCTTTCTGCTGATCCCTTGTCACCCCGAGTGCCAGCCTCAGAATGGC |
| NO: 5) | AGCGTGACATGCTTTGGCCCTGAGGCCGATCAGTGTGTGGCCTGCGCT |
| | CACTACAAGGACCCTCCATTCTGCGTGGCCAGATGTCCTTCTGGCGTG |
| | AAGCCCGACCTGAGCTACATGCCCATCTGGAAGTTCCCCGATGAGGA |
| | AGGCGCCTGCCAGCCTTGTCCTATCAATTGCACACACAGCTGCGTGGA |
| | CCTGGACGACAAAGGATGTCCTGCCGAGCAGAGAGCCTCTCCACTTA |
| | CTGGCGGAGGAAGCGGCGGAGGATCTCCTATCCTGCTGACCTGTCCTA |
| | CAATCAGCATCCTGAGCTTTTTCAGCGTGGCCCTGCTCGTGATCCTGG |
| | CCTGTGTGCTGTGGAAGAAGCGGATCAAGCCCATCGTGTGGCCCAGC |
| | CTGCCTGACCACAAGAAAACCCTGGAACACCTGTGCAAGAAGCCCCG |
| | GAAGAACCTGAACGTGTCCTTCAATCCCGAGAGCTTCCTGGACTGCCA |
| | GATCCACAGAGTGGACGACATCCAGGCCAGAGATGAGGTGGAAGGCT |
| | TTCTGCAGGACACATTCCCTCAGCAGCTGGAAGAGAGCGAGAAGCAG |
| | AGACTCGGCGGAGATGTGCAGAGCCCTAATTGCCCTAGCGAGGACGT |
| | GGTCATCACCCCTGAGAGCTTCGGCAGAGATAGCAGCCTGACATGTCT |
| | GGCCGGCAATGTGTCCGCCTGTGATGCCCCTATCCTGAGCAGCTCCAG |
| | AAGCCTGGATTGCAGAGAGAGCGGCAAGAACGGCCCTCACGTGTACC |
| | AGGATCTGCTCCTGTCTCTGGGCACCACCAATAGCACACTGCCTCCAC |
| | CATTCAGCCTGCAGAGCGGCATCCTGACACTGAACCCTGTTGCTCAGG |
| | GACAGCCCATCCTGACCAGCCTGGGCAGCAATCAAGAAGAGGCCTAC |
| | GTCACCATGAGCAGCTTCTACCAGAACCAGGGCGGAGGCGGATCTGG |
| | TGGTGGTGGATCTTCTCCCGTGGAATGCGACTTCACAAGCCCTGGCGA |
| | CGAGGGACCTCCTAGAAGCTATCTGAGACAGTGGGTCGTGATCCCTCC |
| | ACCTCTGTCTAGTCCTGGACCTCAGGCTTCT |
| CIndR21 | ATGCTGCTGCTGGTCACATCTCTGCTGCTGTGCGAGCTGCCCCATCCT |
| (SEQ ID | GCCTTTCTGCTGATCCCTTGTCACCCCGAGTGCCAGCCTCAGAATGGC |
| NO: 6) | AGCGTGACATGCTTTGGCCCTGAGGCCGATCAGTGTGTGGCCTGCGCT |
| | CACTACAAGGACCCTCCATTCTGCGTGGCCAGATGTCCTTCTGGCGTG |
| | AAGCCCGACCTGAGCTACATGCCCATCTGGAAGTTCCCCGATGAGGA |
| | AGGCGCCTGCCAGCCTTGTCCTATCAATTGCACACACAGCTGCGTGGA |
| | CCTGGACGACAAAGGATGTCCTGCCGAGCAGAGAGCCTCTCCACTTA |
| | CTGGCGGAGGAAGCGGCGGAGGATCTCCTATCCTGCTGACCTGTCCTA |
| | CAATCAGCATCCTGAGCTTTTTCAGCGTGGCCCTGCTCGTGATCCTGG |
| | CCTGTGTGCTGTGGAAGAAGCGGATCAAGCCCATCGTGTGGCCCAGC |
| | CTGCCTGACCACAAGAAAACCCTGGAACACCTGTGCAAGAAGCCCCG |
| | GAAGAACCTGAACGTGTCCTTCAATCCCGAGAGCTTCCTGGACTGCCA |
| | GATCCACAGAGTGGACGACATCCAGGCCAGAGATGAGGTGGAAGGCT |
| | TTCTGCAGGACACATTCCCTCAGCAGCTGGAAGAGAGCGAGAAGCAG |
| | AGACTCGGCGGAGATGTGCAGAGCCCTAATTGCCCTAGCGAGGACGT |
| | GGTCATCACCCCTGAGAGCTTCGGCAGAGATAGCAGCCTGACATGTCT |
| | GGCCGGCAATGTGTCCGCCTGTGATGCCCCTATCCTGAGCAGCTCCAG |
| | AAGCCTGGATTGCAGAGAGAGCGGCAAGAACGGCCCTCACGTGTACC |
| | AGGATCTGCTCCTGTCTCTGGGCACCACCAATAGCACACTGCCTCCAC |
| | CATTCAGCCTGCAGAGCGGCATCCTGACACTGAACCCTGTTGCTCAGG |
| | GACAGCCCATCCTGACCAGCCTGGGCAGCAATCAAGAAGAGGCCTTC |
| | GTCACCATGAGCAGCTTCTACCAGAACCAGGGCGGAGGCGGATCTGG |
| | TGGTGGTGGATCTTCTCCCGTGGAATGCGACTTCACAAGCCCTGGCGA |
| | CGAGGGACCTCCTAGAAGCTATCTGAGACAGTGGGTCGTGATCCCTCC |
| | ACCTCTGTCTAGTCCTGGACCTCAGGCTTCT |
| GMCSF | MLLLVTSLLLCELPHPAFLLIP |
| signal | |
| sequence | |
| (SEQ ID | |
| NO: 7) | |
| Her2tG | CHPECQPQNGSVTCFGPEADQCVACAHYKDPPFCVARCPSGVKPDLSYM |
| extracellular | PIWKFPDEEGACQPCPINCTHSCVDLDDKGCPAEQRASPLTGGGSGGGS |
| domain | |
| (SEQ ID | |
| NO: 8) | |
| IL7R | PILLTCPTISILSFFSVALLVILACVLW |
| transmembr | |
| ane with | |
| dimerizing | |
| insertion | |
| (underlined) | |
| (SEQ ID | |
| NO: 9) | |
| IL7R | KKRIKPIVWPSLPDHKKTLEHLCKKPRKNLNVSFNPESFLDCQIHRVDDIQ |
| intracellular | ARDEVEGFLQDTFPQQLEESEKQRLGGDVQSPNCPSEDVVITPESFGRDSS |
| domain | LTCLAGNVSACDAPILSSSRSLDCRESGKNGPHVYQDLLLSLGTTNSTLPP |
| (SEQ ID | PFSLQSGILTLNPVAQGQPILTSLGSNQEEAYVTMSSFYQNQ |
| NO: 10) | |
| Linker | GGGGSGGGGS |
| (SEQ ID | |
| NO: 11) | |
| IL21R- | SPVECDFTSPGDEGPPRSYLRQWVVIPPPLSSPGPQAS |
| peptide | |
| (SEQ ID | |
| NO: 12) | |
| IL7R | KKRIKPIVWPSLPDHKKTLEHLCKKPRKNLNVSFNPESFLDCQIHRVDDIQ |
| intracellular | ARDEVEGFLQDTFPQQLEESEKQRLGGDVQSPNCPSEDVVITPESFGRDSS |
| domain with | LTCLAGNVSACDAPILSSSRSLDCRESGKNGPHVYQDLLLSLGTTNSTLPP |
| STAT5 | PFSLQSGILTLNPVAQGQPILTSLGSNQEEAFVTMSSFYQNQ |
| signaling | |
| motif | |
| ablated | |
| (underlined) | |
| (SEQ ID | |
| NO: 13) | |
| GMCSF | ATGCTGCTGCTGGTCACATCTCTGCTGCTGTGCGAGCTGCCCCATCCT |
| signal | GCCTTTCTGCTGATCCCT |
| sequence | |
| (SEQ ID | |
| NO: 14) | |
| Her2tG | TGTCACCCCGAGTGCCAGCCTCAGAATGGCAGCGTGACATGCTTTGGC |
| extracellular | CCTGAGGCCGATCAGTGTGTGGCCTGCGCTCACTACAAGGACCCTCCA |
| domain | TTCTGCGTGGCCAGATGTCCTTCTGGCGTGAAGCCCGACCTGAGCTAC |
| (SEQ ID | ATGCCCATCTGGAAGTTCCCCGATGAGGAAGGCGCCTGCCAGCCTTGT |
| NO: 15) | CCTATCAATTGCACACACAGCTGCGTGGACCTGGACGACAAAGGATG |
| | TCCTGCCGAGCAGAGAGCCTCTCCACTTACTGGCGGAGGAAGCGGCG |
| | GAGGATCT |
| IL7R | CCTATCCTGCTGACCTGTCCTACAATCAGCATCCTGAGCTTTTTCAGCG |
| transmembr | TGGCCCTGCTCGTGATCCTGGCCTGTGTGCTGTGG |
| ane with | |
| dimerizing | |
| insertion | |
| (underlined) | |
| SEQ ID | |
| NO: 16) | |
| IL7R | AAGAAGCGGATCAAGCCCATCGTGTGGCCCAGCCTGCCTGACCACAA |
| intracellular | GAAAACCCTGGAACACCTGTGCAAGAAGCCCCGGAAGAACCTGAACG |
| domain | TGTCCTTCAATCCCGAGAGCTTCCTGGACTGCCAGATCCACAGAGTGG |
| (SEQ ID | ACGACATCCAGGCCAGAGATGAGGTGGAAGGCTTTCTGCAGGACACA |
| NO: 17) | TTCCCTCAGCAGCTGGAAGAGAGCGAGAAGCAGAGACTCGGCGGAGA |
| | TGTGCAGAGCCCTAATTGCCCTAGCGAGGACGTGGTCATCACCCCTGA |
| | GAGCTTCGGCAGAGATAGCAGCCTGACATGTCTGGCCGGCAATGTGT |
| | CCGCCTGTGATGCCCCTATCCTGAGCAGCTCCAGAAGCCTGGATTGCA |
| | GAGAGAGCGGCAAGAACGGCCCTCACGTGTACCAGGATCTGCTCCTG |
| | TCTCTGGGCACCACCAATAGCACACTGCCTCCACCATTCAGCCTGCAG |
| | AGCGGCATCCTGACACTGAACCCTGTTGCTCAGGGACAGCCCATCCTG |
| | ACCAGCCTGGGCAGCAATCAAGAAGAGGCCTACGTCACCATGAGCAG |
| | CTICTACCAGAACCAG |
| Linker | GGCGGAGGCGGATCTGGTGGTGGTGGATCT |
| (SEQ ID | |
| NO: 18) | |
| IL21R- | TCTCCCGTGGAATGCGACTTCACAAGCCCTGGCGACGAGGGACCTCCT |
| peptide | AGAAGCTATCTGAGACAGTGGGTCGTGATCCCTCCACCTCTGTCTAGT |
| (SEQ ID | CCTGGACCTCAGGCTTCT |
| NO: 19) | |
| IL7R | AAGAAGCGGATCAAGCCCATCGTGTGGCCCAGCCTGCCTGACCACAA |
| intracellular | GAAAACCCTGGAACACCTGTGCAAGAAGCCCCGGAAGAACCTGAACG |
| domain with | TGTCCTTCAATCCCGAGAGCTTCCTGGACTGCCAGATCCACAGAGTGG |
| STATS | ACGACATCCAGGCCAGAGATGAGGTGGAAGGCTTTCTGCAGGACACA |
| signaling | TTCCCTCAGCAGCTGGAAGAGAGCGAGAAGCAGAGACTCGGCGGAGA |
| motif | TGTGCAGAGCCCTAATTGCCCTAGCGAGGACGTGGTCATCACCCCTGA |
| ablated | GAGCTTCGGCAGAGATAGCAGCCTGACATGTCTGGCCGGCAATGTGT |
| (underlined) | CCGCCTGTGATGCCCCTATCCTGAGCAGCTCCAGAAGCCTGGATTGCA |
| (SEQ ID | GAGAGAGCGGCAAGAACGGCCCTCACGTGTACCAGGATCTGCTCCTG |
| NO: 20) | TCTCTGGGCACCACCAATAGCACACTGCCTCCACCATTCAGCCTGCAG |
| | AGCGGCATCCTGACACTGAACCCTGTTGCTCAGGGACAGCCCATCCTG |
| | ACCAGCCTGGGCAGCAATCAAGAAGAGGCCTTCGTCACCATGAGCAG |
| | CTICTACCAGAACCAG |
| MND | GAACAGAGAAACAGGAGAATATGGGCCAAACAGGATATCTGTGGTA |
| promoter | AGCAGTTCCTGCCCCGGCTCAGGGCCAAGAACAGTTGGAACAGCAGA |
| (SEQ ID | ATATGGGCCAAACAGGATATCTGTGGTAAGCAGTTCCTGCCCCGGCTC |
| NO: 21) | AGGGCCAAGAACAGATGGTCCCCAGATGCGGTCCCGCCCTCAGCAGT |
| | TTCTAGAGAACCATCAGATGTTTCCAGGGTGCCCCAAGGACCTGAAAT |
| | GACCCTGTGCCTTATTTGAACTAACCAATCAGTTCGCTTCTCGCTTCTG |
| | TTCGCGCGCTTCTGCTCCCCGAGCTCTATATAAGCAGAGCTCGTTTAG |
| | TGAACCGTCAGATC |
| RBG PolyA | TTTTCCCTCTGCCAAAAATTATGGGGACATCATGAAGCCCCTTGAGCA |
| (SEQ ID | TCTGACTTCTGGCTAATAAAGGAAATTTATTTTCATTGCAATAGTGTGT |
| NO: 22) | TGGAATTTTTTGTGTCTCTCACTCGGAAGGACATAAGG |
| Linker | GGG |
| (SEQ ID | |
| NO: 23) | |
| Linker | GGGGS |
| (SEQ ID | |
| NO: 24) | |
| Linker | GGGGSGGGGS |
| (SEQ ID | |
| NO: 25) | |
| Linker | GGGGSGGGGSGGGGS |
| (SEQ ID | |
| NO: 26) | |
| Anti- | MLLLVTSLLLCELPHPAFLLIPEVQLVESGGGLVQPGRSLRLSCAASGFTF |
| CD19CAR | DDYAMHWVRQAPGKGLEWVSGISWNSGRIGYADSVKGRFTISRDNAKN |
| (SEQ ID | SLFLQMNSLRAEDTAVYYCARDQGYHYYDSAEHAFDIWGQGTVVTVSS |
| NO: 27) | GGGGSGGGGSGGGGSQSALTQPRSVSGFPGQSVTISCTGTTSDDVSWYQ |
| | QHPGKAPQLMLYDVSKRPSGVPHRFSGSRSGRAASLIISGLQTEDEADYF |
| | CSSYAGRYNSVLFGGGTKLTVLESKYGPPCPPCPMFWVLVVVGGVLACY |
| | SLLVTVAFIIFWVKRGRKKLLYIFKQPFMRPVQTTQEEDGCSCRFPEEEEG |
| | GCELRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQ |
| | GLSTATKDTYDALHMQALPPR |
| CD19t | MPPPRLLFFLLFLTPMEVRPEEPLVVKVEEGDNAVLQCLKGTSDGPTQQL |
| (SEQ ID | TWSRESPLKPFLKLSLGLPGLGIHMRPLAIWLFIFNVSQQMGGFYLCQPGP |
| NO: 28) | PSEKAWQPGWTVNVEGSGELFRWNVSDLGGLGCGLKNRSSEGPSSPSGK |
| | LMSPKLYVWAKDRPEIWEGEPPCVPPRDSLNQSLSQDLTMAPGSTLWLS |
| | CGVPPDSVSRGPLSWTHVHPKGPKSLLSLELKDDRPARDMWVMETGLLL |
| | PRATAQDAGKYYCHRGNLTMSFHLEITARPVLWHWLLRTGGWKVSAVT |
| | LAYLIFCLCSLVGILHLQRALVLRRKR |
| anti- | ATGCTGCTGCTGGTCACATCTCTGCTGCTGTGCGAGCTGCCCCATCCT |
| CD19CAR | GCCTTTCTGCTGATTCCTGAGGTGCAGCTGGTGGAATCTGGCGGAGGA |
| SEQ ID | CTTGTGCAGCCTGGCAGAAGCCTGAGACTGTCTTGTGCCGCCAGCGGC |
| NO: 29) | TTCACCTTCGACGATTATGCCATGCACTGGGTCCGACAGGCCCCTGGC |
| | AAAGGACTTGAATGGGTGTCCGGCATCAGCTGGAACAGCGGCAGAAT |
| | CGGCTATGCCGACAGCGTGAAGGGCAGATTCACCATCAGCCGGGACA |
| | ACGCCAAGAACAGCCTGTTCCTGCAGATGAACTCCCTGAGAGCCGAG |
| | GACACCGCCGTGTACTACTGTGCCAGAGATCAGGGCTACCACTACTAC |
| | GACTCTGCCGAGCACGCCTTCGATATCTGGGGCCAGGGAACAGTGGT |
| | CACCGTTTCTAGCGGAGGCGGAGGATCTGGTGGTGGTGGCTCTGGTG |
| | GCGGCGGATCTCAATCTGCTCTGACCCAGCCTAGAAGCGTGTCCGGCT |
| | TTCCTGGCCAGAGCGTGACAATCAGCTGTACCGGCACCACCAGCGAC |
| | GACGTGTCATGGTATCAGCAGCACCCTGGAAAGGCCCCTCAGCTCAT |
| | GCTGTACGATGTGTCCAAAAGACCCAGCGGCGTGCCCCACAGATTTTC |
| | CGGAAGTAGATCTGGCAGAGCCGCCAGCCTGATCATCTCTGGACTGC |
| | AGACAGAGGACGAGGCCGACTACTTCTGCAGCAGCTATGCCGGCAGA |
| | TACAACAGCGTGCTGTTTGGCGGAGGCACCAAGCTGACCGTGCTGGA |
| | ATCTAAGTACGGCCCTCCTTGTCCTCCATGTCCTATGTTCTGGGTGCTC |
| | GTGGTTGTTGGCGGAGTGCTGGCCTGTTACAGCCTGCTGGTTACCGTG |
| | GCCTTCATCATCTTTTGGGTCAAGCGGGGCAGAAAGAAGCTGCTCTAC |
| | ATCTTCAAGCAGCCCTTCATGCGGCCCGTGCAGACCACACAAGAGGA |
| | AGATGGCTGCAGCTGTCGGTTCCCCGAGGAAGAAGAAGGCGGCTGCG |
| | AGCTGAGAGTGAAGTTCAGCAGATCCGCTGACGCCCCTGCCTATCAG |
| | CAGGGACAGAACCAGCTGTATAACGAGCTGAACCTGGGGCGCAGAGA |
| | AGAGTACGACGTGCTGGACAAGAGAAGAGGCAGGGACCCTGAGATG |
| | GGCGGCAAGCCCAGAAGAAAGAACCCTCAAGAGGGCCTCTACAACG |
| | AGCTGCAGAAAGACAAGATGGCCGAGGCCTACAGCGAGATCGGAAT |
| | GAAGGGCGAACGCAGAAGAGGAAAGGGCCACGACGGACTGTATCAG |
| | GGACTGAGCACCGCCACCAAGGACACCTATGATGCCCTGCACATGCA |
| | GGCCCTGCCTCCAAGA |
| CD19t | ATGCCACCTCCTCGCCTCCTCTTCTTCCTCCTCTTCCTCACCCCCATGG |
| (SEQ ID | AAGTCAGGCCCGAGGAACCTCTAGTGGTGAAGGTGGAAGAGGGAGAT |
| NO: 30) | AACGCTGTGCTGCAGTGCCTCAAGGGGACCTCAGATGGCCCCACTCA |
| | GCAGCTGACCTGGTCTCGGGAGTCCCCGCTTAAACCCTTCTTAAAACT |
| | CAGCCTGGGGCTGCCAGGCCTGGGAATCCACATGAGGCCCCTGGCCA |
| | TCTGGCTTTTCATCTTCAACGTCTCTCAACAGATGGGGGGCTTCTACCT |
| | GTGCCAGCCGGGGCCCCCCTCTGAGAAGGCCTGGCAGCCTGGCTGGA |
| | CAGTCAATGTGGAGGGCAGCGGGGAGCTGTTCCGGTGGAATGTTTCG |
| | GACCTAGGTGGCCTGGGCTGTGGCCTGAAGAACAGGTCCTCAGAGGG |
| | CCCCAGCTCCCCTTCCGGGAAGCTCATGAGCCCCAAGCTGTATGTGTG |
| | GGCCAAAGACCGCCCTGAGATCTGGGAGGGAGAGCCTCCGTGTGTCC |
| | CACCGAGGGACAGCCTGAACCAGAGCCTCAGCCAGGACCTCACCATG |
| | GCCCCTGGCTCCACACTCTGGCTGTCCTGTGGGGTACCCCCTGACTCT |
| | GTGTCCAGGGGCCCCCTCTCCTGGACCCATGTGCACCCCAAGGGGCCT |
| | AAGTCATTGCTGAGCCTAGAGCTGAAGGACGATCGCCCTGCCAGAGA |
| | TATGTGGGTAATGGAGACGGGTCTGTTGTTGCCCCGGGCCACAGCTCA |
| | AGACGCTGGAAAGTATTATTGTCACCGTGGCAACCTGACCATGTCATT |
| | CCACCTGGAGATCACTGCTCGGCCAGTACTATGGCACTGGCTGCTGAG |
| | GACTGGTGGCTGGAAGGTCTCAGCTGTGACTTTGGCTTATCTGATCTT |
| | CTGCCTGTGTTCCCTTGTGGGCATTCTTCATCTTCAAAGAGCCCTGGTC |
| | CTGAGGAGGAAAAGA |

### EXAMPLES

Adoptive T cell therapy relies upon persistence and functional durability of administered T cells. Environmental signals received by the T cell, including cytokine inputs, contribute heavily to these behaviors. As described herein, CART cells supplemented with IL-7 stimulation showed greater long-term persistence, whereas IL-21 stimulation led to enhanced cytotoxic potency and functional durability. It was hypothesized that combining these two interleukin signaling inputs would have complementary effects on CAR T cell functionality. Herein, is presented a novel chimeric cytokine receptor design capable of providing signaling outputs of both IL-7 and IL-21. These studies examined CAR T cell survival, proliferation, cytotoxic potency, and anti-tumor capacity in the context of long-term co-cultures with tumor cells. It was found that CAR T cells equipped with the hybrid IL-7 IL-21 signaling cytokine receptor demonstrated extended persistence and functional durability. These attributes make the hybrid cytokine receptor a promising candidate to supplement CAR T cell therapy, particularly in the solid tumor space, where the microenvironment contributes to T cell deactivation, Concurrently, transcriptional regulation systems were explored to allow spatiotemporal control of hybrid cytokine receptor expression such that it can be implemented in a clinically viable manner.

### Example 1—Design of the Cytokine-Independent Receptor for Signaling

Disclosed herein is a non-limiting example of a hybrid IL7 and I121 cytokine receptor for combinatorial interleukin signaling. The cytokine independent receptor for IL7 signaling (ClndR7) was designed based on a naturally occurring mutant of IL7R with an insertion in the transmembrane domain commonly found in leukemias. The resultant IL7R homodimers signal independently of cytokine and gamma chain.

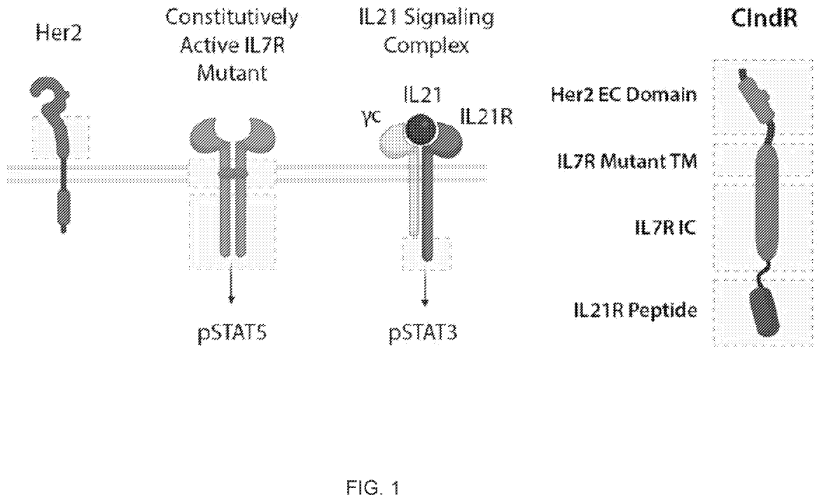

FIG. 1 (left panel) depicts a schematic which includes a HER polypeptide with a portion of the extracellular highlighted within a dotted box; a constitutively active IL7R mutant with a transmembrane domain containing a mutation and an intracellular STAT5 signaling domain each highlighted in dotted boxes; and an IL21 signaling complex with a STAT3 signaling domain highlight in a dotted box. FIG. 1 (right panel) is a schematic which shows an example chimeric receptor (CIndR) which includes the extracellular domain of the HER2; the transmembrane domain containing a mutation and the intracellular STAT5 signaling domain of the IL7R; and the signaling domain of the IL21 signaling complex joined to the rest of the chimeric receptor via a linker. From N-terminus to C-terminus, the cytokine-independent receptor (CIndR) incorporates: (1) an extracellular domain of Her2 for cell surface detection, (2) a mutant version of the IL7R transmembrane for constitutive signaling, (3) the IL7R intracellular domain, and (4) an IL21R peptide tethered via a flexible linker. The IL7R intracellular domain contains a STAT5-activating motif and the IL21R peptide contains a STAT3-activating motif.

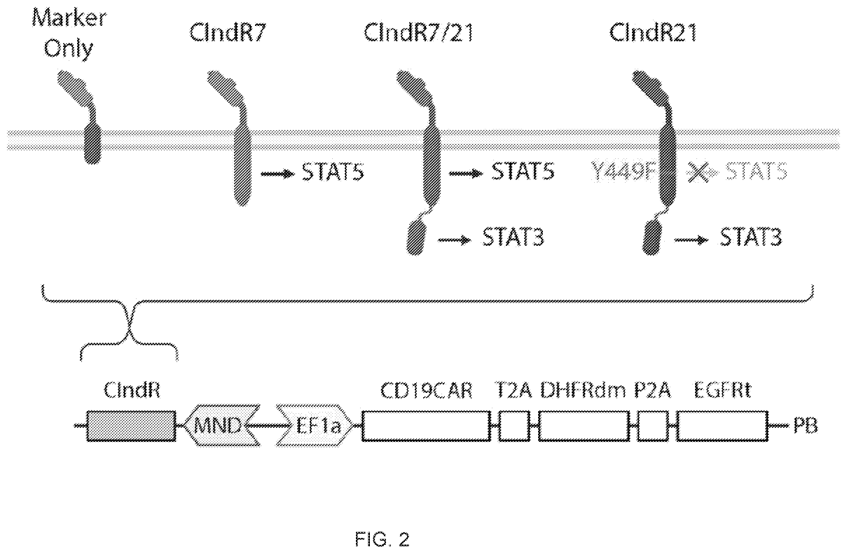

FIG. 2 (upper panel) depicts a schematic which includes various chimeric receptors, and a control marker only receptor. Three varieties of CIndR were created with distinct intracellular designs: 1) ClndR7, incorporating only the IL7R intracellular domain, 2) ClndR7/21, incorporating the IL21R peptide and the IL7R intracellular domain, and 3) ClndR21, incorporating the IL21R peptide and the IL7R intracellular domain with the STAT5-activating tyrosine residue mutated to a phenylalanine to ablate STAT5 signaling. Polynucleotides encoding the various CIndR were cloned into dual-promoter constructs which also included polynucleotides encoding an anti-CD19 CAR, selectable marker (DHFRdm), and a cell surface marker (EGFRt). FIG. 2 (lower panel) depicts a polynucleotide encoding a CIndR linked to a MND promoter; an EF1-alpha promoter linked to a polynucleotide encoding the anti-CD19 CAR, a T2A sequence, the selectable marker (DHFRdm), a P2A sequence, and the cell surface marker (EGFRt) in a vector.

Example chimeric receptors are depicted in FIGS. 3A-3D. FIG. 3A depicts a polypeptide comprising: (1) an extracellular domain of Her2tG, (2) a transmembrane domain of IL7R with an insertion, and (3) an intracellular domain of IL7R. FIG. 3B depicts a ClndR7 which also includes a mutation Y449 in the IL7R domain which can promote formation of a disulfide bridge between IL7R monomers. The chimeric receptor, ClndR7/21, was designed by appending a peptide (5) from the IL21 receptor onto the C-terminus of ClndR7 via a flexible linker (4) (FIG. 3C) and a version included the a mutation Y449 in the IL7R domain (FIG. 3D). The design was further iterated on to generate ClndR21 by ablating the STAT5 signaling motif in the IL7 receptor cytoplasmic domain (FIG. 3E).

### Example 2—Proof of Concept for Her2tG as a Surface Marker for CIndR7

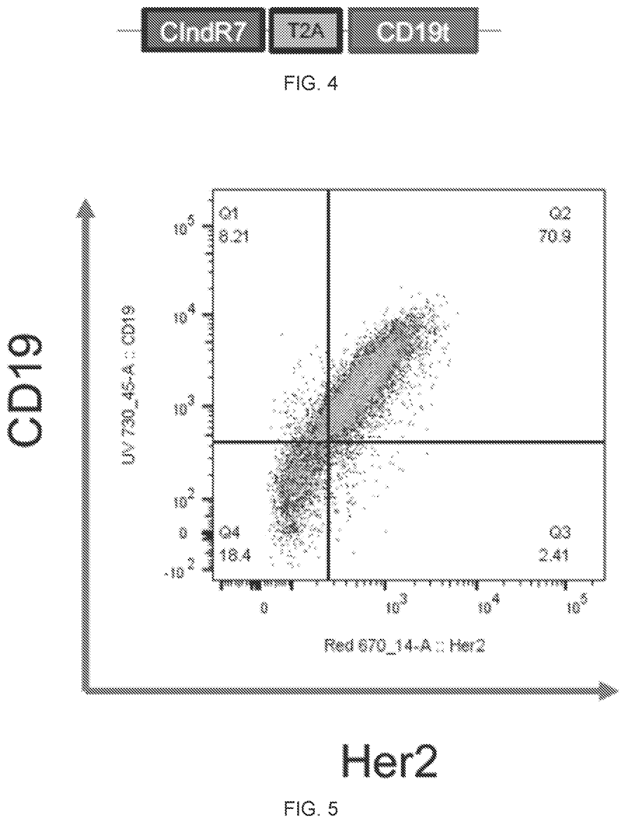

To determine whether a truncated HER2 (Her2tG) polypeptide can be used as a surface marker to test for ClndR7, a plasmid containing a truncated CD19 polypeptide (CD19t), ClndR7, and a T2A was introduced into T cells (FIG. 4). The T2A ensured that the CD19t and ClndR7 were translated into two separate proteins. As measured by FACS, Her2 staining was directly proportional to CD19 staining in cells (FIG. 5) This flow cytometric scatterplot demonstrated that Her2tG served as a surface marker for ClndR7 expression, as it correlated with the presence of a co-expressed truncated CD19 marker protein.

### Example 3—Production of CIndR-Expressing CAR T Cells and Enrichment

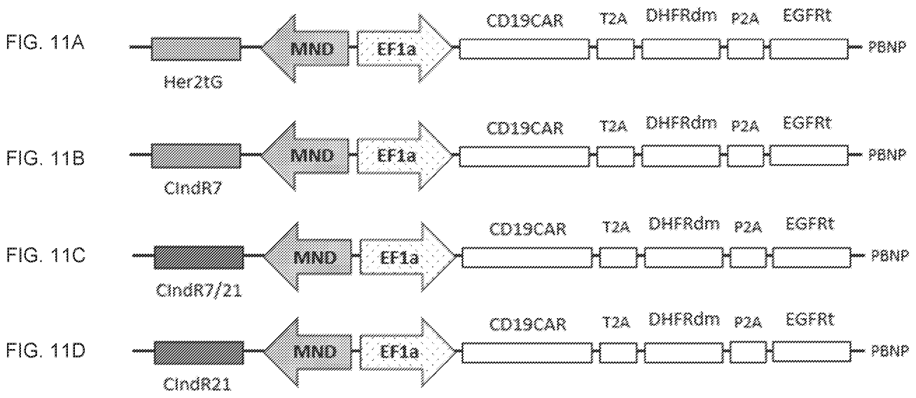

CIndR sequences were introduced into a PiggyBac nanoplasmid construct as self-contained cistrons with expression driven by an MIND promoter and terminated by a RBG polyadenylation sequence. The constructs also contained a separate cistron coding for a human anti-CD19 CAR, a double mutant of the drug resistance gene dihydrofolate reductase (DHFRdm), and a surface marker protein in the form of truncated epidermal growth factor polypeptide (EGFRt) (FIGS. 11A-i ID). The Her2tG cargo served as a non-active control.

These CIndR-encoding constructs were introduced into CD4⁺ and CD8⁺ T cells via electroporation alongside mRNA encoding for the PiggyBac transposon to allow for stable integration into the genome. T cells with stably integrated transgenic sequences were enriched through a combination of CAR-specific activation and drug selection with methotrexate, resistance to which is conferred by DHFRdm protein expression.

### Example 4—Flow Cytometry for the Detection of Phosphorylated STAT Proteins

T cells were removed from culture, washed twice with PBS to remove supplemented cytokines, and returned to culture overnight in the absence supplemental cytokines. The following day, cells were either left un-treated, or treated with IL-7 (5 ng/mL) or IL-21 (5 ng/mL) for 20 minutes. The cells were then fixed using BD CytoFix reagent, permeabilized using BD Perm Buffer 111, stained using antibodies specific for phosphorylated STAT5 (pSTAT5) and phosphorylated STAT3 (pSTAT3), and finally analyzed by flow cytometry.

### Example 4—Hybrid Cytokine Receptors Recapitulate IL-7 and IL-21 Signaling in Human T Cells

CD19CAR T cells expressing CIndR variants were characterized by flow cytometry with antibodies specific for phosphorylated STAT3. Controls included CAR T cells expressing marker only and treated with either exogenous IL-7 or IL-21.

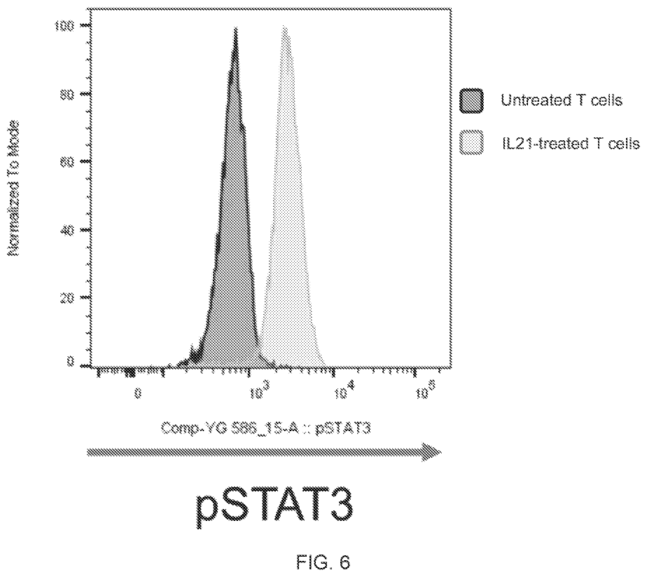

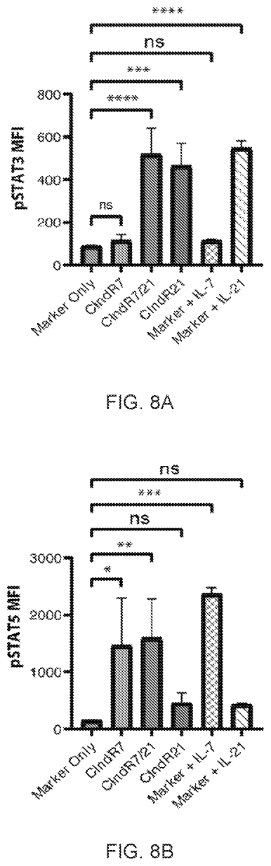

Controls contacted with IL-21 or T cell groups expressing a CIndR with the IL21R peptide displayed activation of STAT3 (FIG. 6, FIG. 8A). This demonstrated that the IL21R signaling domain incorporated into ClndR7/21 and ClndR21 was functioning and provided STAT3 activation in the same pattern as exposure to exogenous IL-21.

Controls contacted with IL-7 or T cell groups expressing a CIndR with a non-mutated IL7R intracellular domain displayed activation of STAT5 (FIG. 8B). This demonstrated that the IL7R signaling domain incorporated into ClndR7 and ClndR7/21 was functioning provided STAT5 activation in the same pattern as exposure to exogenous IL-7

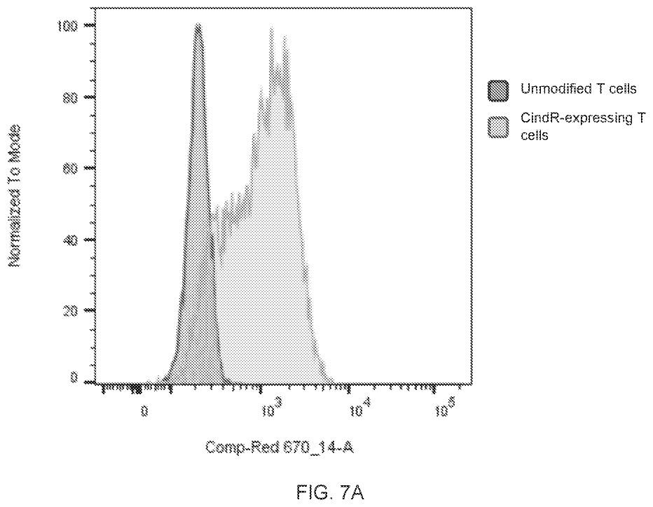

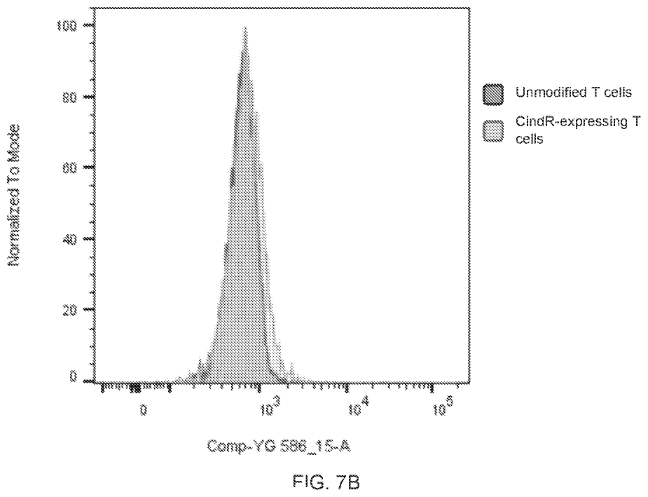

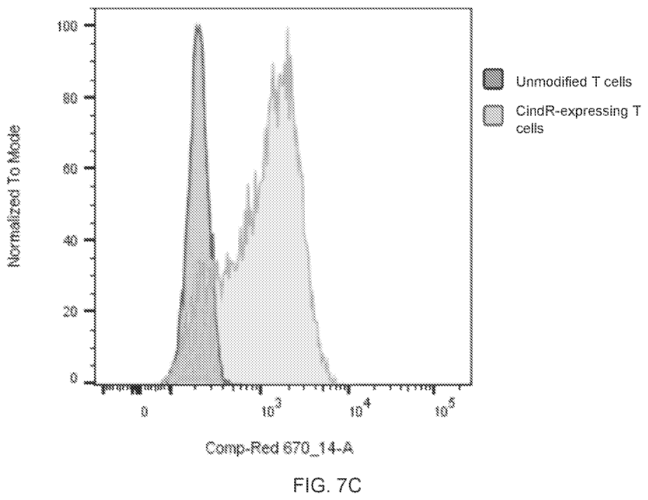

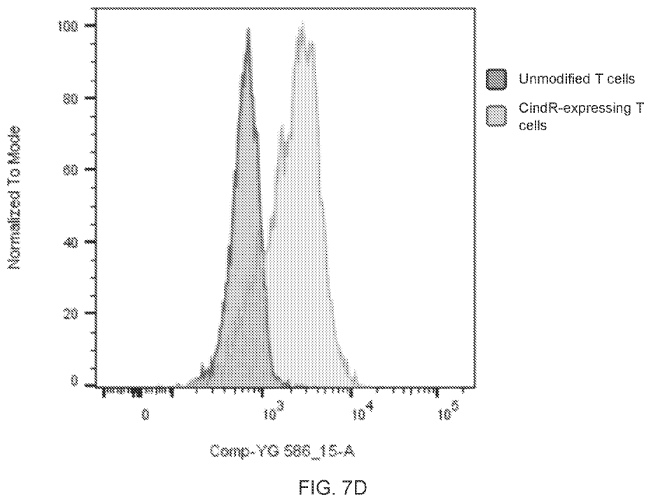

In contrast, ClndR7 expression by human CD8+ T cells led to an increase in phosphorylated STAT5 (pSTAT5) (FIG. 7A), but minimal phosphorylation of STAT3 (FIG. 7B). ClndR7/21 expression, however, led to robust phosphorylation of STAT3 (FIG. 7D), while maintaining high pSTAT5 levels (FIG. 7C). Overall, ClndR7-expressing T cells showed significantly higher levels of pSTAT5; ClndR21-expressing T cells showed significantly higher levels pSTAT3; and ClndR7/21-expressing T cells showed significantly higher levels of both pSTAT3 and pSTAT5. This demonstrated that the panel of CIndR proteins conferred STAT3 and STAT5 signaling activity both in isolation and in combination.

### Example 5—I21R-Peptide Linker Length

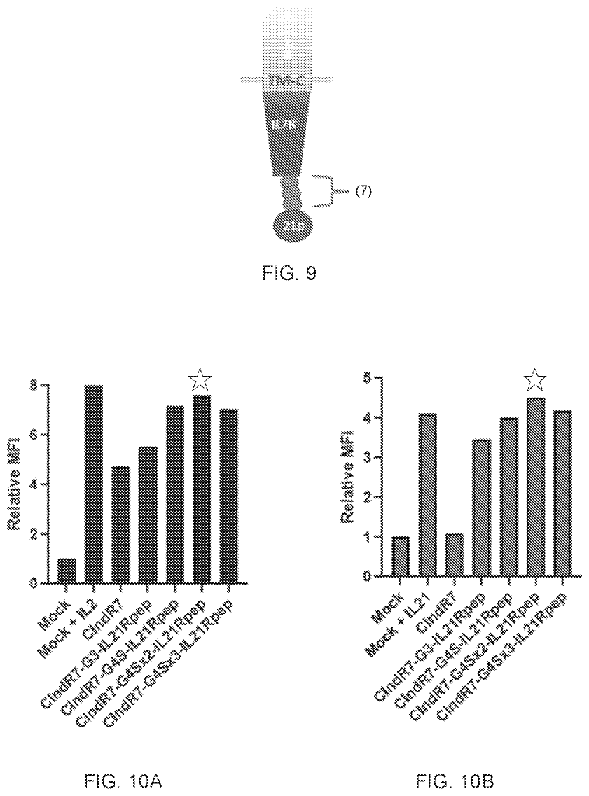

The linker (FIG. 7) between the IL7R cytoplasmic domain and the IL21R-peptide was tested to provide the highest STAT3 and STAT5 signaling (FIG. 9). The signaling capabilities were tested for polypeptides containing various flexible linker candidates: GGG (SEQ. ID. NO: 23), GGGGS (SEQ. ID. NO: 24), (GGGGS)₂ (SEQ. ID. NO: 25) or (GGGGS)₃ (SEQ. ID. NO: 26). Flow cytometric analysis showed that the double quadruple glycine+serine linker ((GGGGS)₂ (SEQ. ID. NO: 25)) showed the highest STAT3 and STAT5 activation, so this linker length was incorporated into all constructs for further characterization (FIGS. 10A-10B).

### Example 6—In Vitro Anti-Tumor Activity Assays

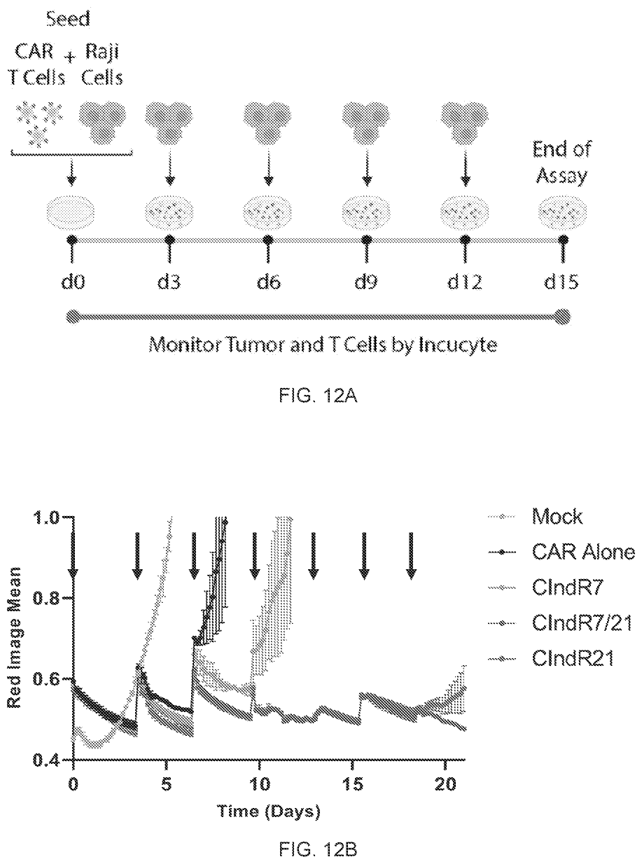

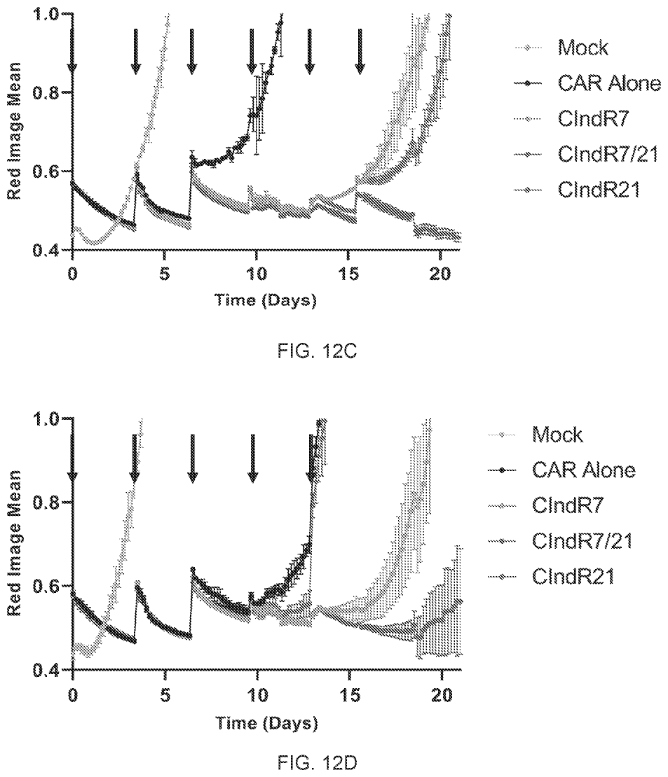

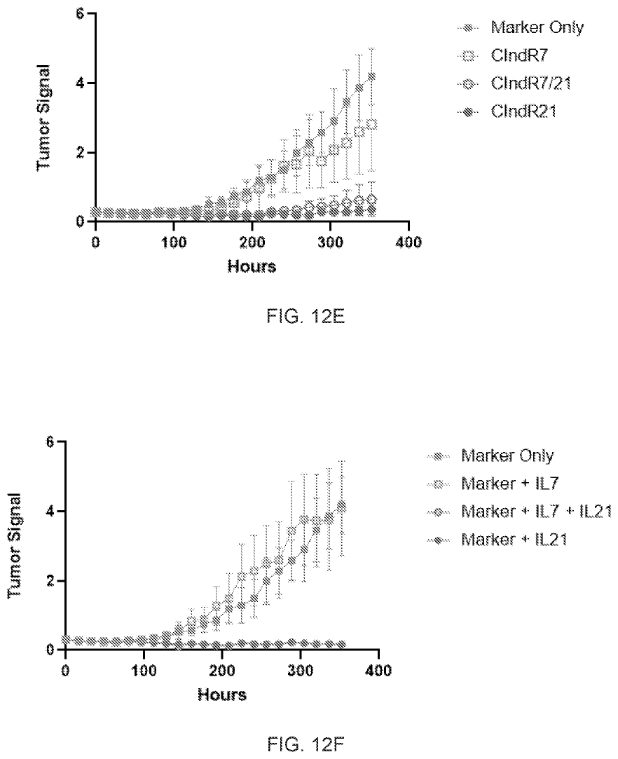

Long-term anti-tumor activity of T cells containing CindR and an anti-CD19 CAR was examined using a three-week repetitive tumor challenge assay. A summary of the experimental approach is shown in FIG. 12A. Briefly, CD4⁺ and CD8⁺ (effector cells, anti-CD19 CAR T cells) were mixed at a 1 to 1 ratio and then co-cultured with human lymphoma Raji cells (CD19+ target cells) at a 2 to 1 effector to target ratio. The Raji cells had been modified to express the red fluorescent protein mCherry, and an Incucyte S3 instrument performed live cell fluorescent imaging to track tumor presence over time. Raji cells were added to the co-culture at the same dose every three days until separation in tumor presence emerged between each experimental T cell group. Live imaging was used to quantify ability of CAR T cells to suppress tumor growth over a fifteen-day period. The amount of red fluorescent signal in each T cell group was quantified across three different donors.

As shown in FIGS. 12B-12D, arrows indicate when Raji tumor cells were added to the co-culture. Overall, CAR T cell containing the CIndRs had an extended anti-tumor activity compared to CAR T cells lacking the CIndRs. CIndR-7/21 showed the greatest ability to suppress tumor growth across all three donors.

FIG. 12E and FIG. 12F depict an additional experiment in which CAR T cells expressing IL-21 signaling CIndRs (CIndR7/21 and ClndR21) were able to drastically suppress tumor growth compared to other groups (FIG. 12E). Of note, the same pattern of tumor suppression was observed when marker control CAR T cells were exposed to exogenous cytokines—only IL-21 treated control CAR T cells were able to suppress tumor growth (FIG. 12F). These findings further showed that IL-21-signaling hybrid cytokine receptors improved CAR T cell anti-tumor activity under prolonged tumor exposure and that this effect recapitulated the advantage provided by exposure to exogenous IL-21

### Example 7—Hybrid Cytokine Receptors Increase CAR T Cell Proliferation

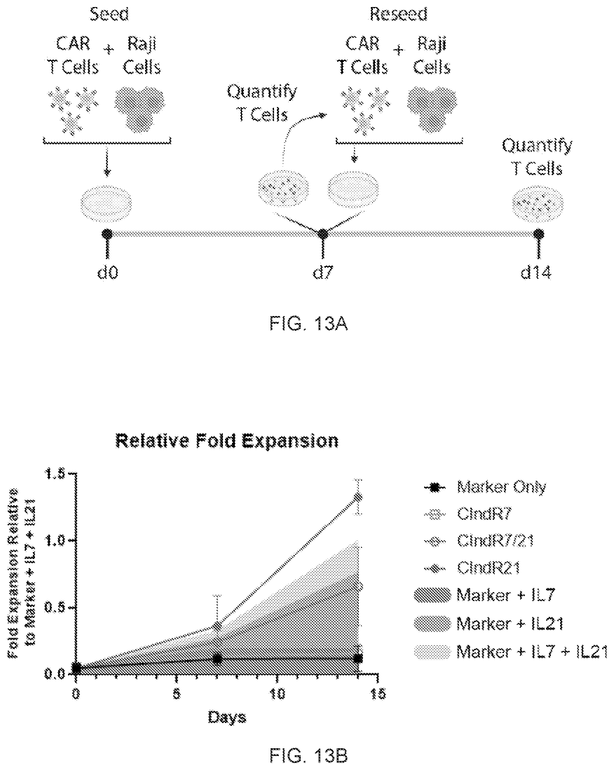

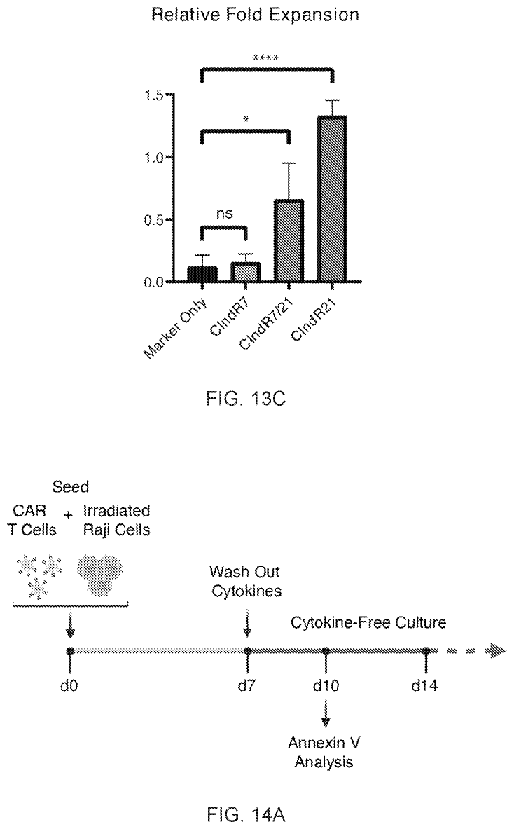

The ability of CIndRs to augment CAR T cell proliferation was assessed using an experimental approach summarized in FIG. 13A. Briefly, CAR T cell groups were co-cultured with Raji target cells on day 0. On day 7, T cells were quantified and reseeded with a fresh batch of Raji target cells. On day 14, T cells were quantified again, and T cell expansion over the two-week period was calculated. Controls included marker only CAR T cells cultured with exogenous IL-7, IL-21, or IL-7 and IL-21.

The fold expansion of each CAR T cell group was calculated relative to the maximum expansion achieved by the IL-7 and IL-21 contacted control group on an individual donor basis. The relative fold expansion values over time are shown in FIG. 13B, and final relative fold expansion values are shown in FIG. 13C. The relative fold expansion revealed that IL-21 signaling receptors provided a drastic improvement to CAR T cell expansion, most notably upon the second exposure to Raji tumor cells. Moreover, these findings demonstrated that the IL-21 signaling CIndRs increased CAR T cell proliferation in response to antigen stimulation to a statistically significant degree.

### Example 8—Hybrid Cytokine Receptors Suppress Apoptosis

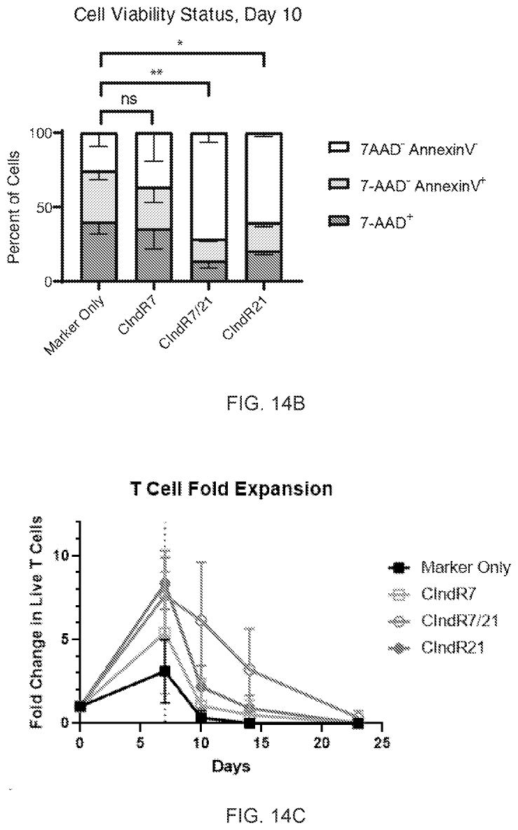

The ability of CIndRs to suppress CAR T cell apoptosis and trigger cytokine-independent growth was assessed using an experimental approach summarized in FIG. 14A. Briefly, CAR T groups were co-cultured with irradiated Raji target cells on day 0. On day 7, the culture was washed to remove any cytokines from culture media, and T cells were reseeded and monitored thereafter for cytokine-free growth. On day 10, T cells were taken for viability status evaluation by Annexin V binding flow cytometry analysis. The cell viability status was determined by a flow cytometry experiment conducted on day 10 incorporating Annexin V binding and the 7-AAD viability dye absorption by the T cell population. Annexin V binds to pre-apoptotic cells and 7-AAD is incorporated into dead cells, allowing for a quantification of healthy cells (Annexin V⁻ 7-AAD⁻), pre-apoptotic cells (Annexin V⁺ 7-AAD⁻), and dead cells (7-AAD⁻).

Statistical analysis revealed that IL-21 signaling CIndRs provided a significant reduction in T cell apoptosis compared to the marker control group (FIG. 14B). T cell growth was monitored after cytokine withdrawal on day 7, As shown in FIG. 14C, while CIndRs extended CAR T cell survival, they did not lead to cytokine-independent T cell expansion in the absence of antigen.

### Example 9—IL-21-Signaling Receptors Sustain a Subset of Stem Cell Memory (CAR T Cells

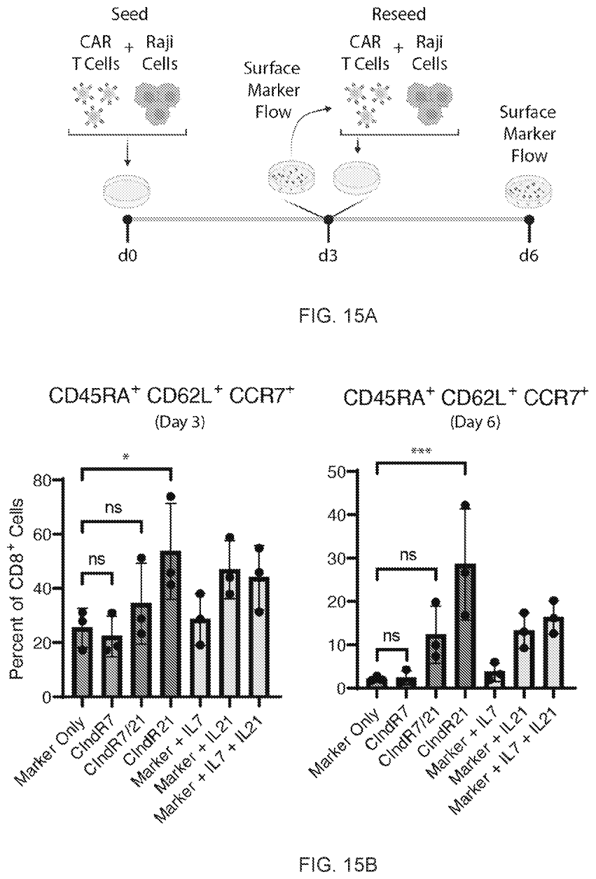

The effect of CIndRs on CAR T cell differentiation marker expression was assessed using an experimental approach summarized in FIG. 15A. Briefly, CAR T groups were co-cultured with Raji target cells on day 0. On day 3, T cells were characterized for differentiation marker expression by flow cytometry, quantified and reseeded with a fresh batch of Raji target cells. On day 6, T cells were again characterized for differentiation marker expression by flow cytometry.

As shown in FIG. 15B, CAR T cells equipped with IL-21 signaling CIndRs showed a greater proportion of T cells expressing markers of stem cell memory T cells. This effect was most pronounced and statistically significant for the ClndR21-expressing CART cell group, and the difference between this group and the control T cell group widened between the first and second target cell exposure. A similar pattern was observed among control marker only CAR T cell groups exposed to exogenous IL-21. These findings reveal that IL-21-signaling receptors sustained a sub-population of stem cell memory T cells.

### Example 10—IL-21-Signaling Receptors Enhance Tumor Suppression in Leukemia-Bearing Mice

The ability ClndRs to improve CAR T cell tumor suppression in vivo was assessed using a systemic leukemia model in (NSG) immunodeficient mice. Briefly, 11-13 week old female mice were systemically injected with CD19+ human Nalm-6 leukemia cells on day 0. Nalm-6 cells were engineered to express the firefly luciferase bioluminescent reporter gene, and mice were monitored for tumor presence by luminescent imaging. On day 7, mice were systemically injected with human T cells from five experimental treatment groups: (1) unmodified T cells; (2) CD19CAR T cells co-expressing marker only; (3) CD19CAR T cells co-expressing CIndR7; (4) CD19CAR T cells co-expressing ClndR7/21; and (5 CD19CAR T cells co-expressing ClndR21.

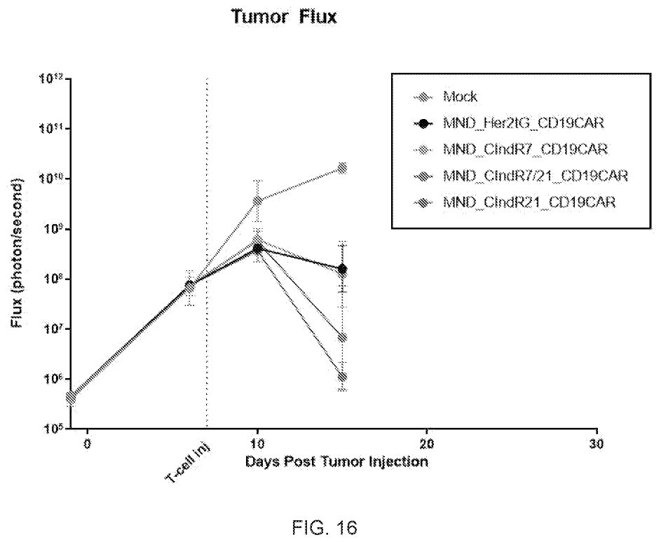

Bioluminescent quantification of tumor presence revealed that mice treated with any CD19CAR T cell group showed a reduction in tumor presence compared to mice treated with unmodified T cells by day 15 (FIG. 16). However, mice treated with CD19CAR T cells which also contained IL-21 signaling CIndRs showed drastically lower tumor presence at day 15. Moreover, some such mice showed almost complete tumor elimination. This suggested that the IL-21 signaling functionality imparted by the hybrid cytokine receptor technology described herein potentiates CAR T cells to be more effective anti-cancer therapeutic agents.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A chimeric cytokine receptor comprising:
(i) an extracellular domain,
optionally, wherein the extracellular domain comprises a cell surface selectable polypeptide, optionally, wherein the selectable polypeptide is selected from an extracellular HER2 domain, an extracellular EGFR domain, and an extracellular CD19 domain;
(ii) a transmembrane interleukin-7 receptor (IL-7R) domain; and
(iii) an intracellular IL-7R domain.

2-85. (canceled)
